(19) United States
(12) Patent Application Publication
(10) Pub. No.: US 2005/0119246 A1
(43) Pub. Date: Jun. 2, 2005

(54) 1H-pyrazolyl derivative compounds, for use in diseases associated with the 5-ht2c receptor

(75) Inventors: Gaetan H. Ladouceur, Guilford, CT (US); Emil Velthuisen, Santa Ana, CA (US); Soongyu Choi, Skillman, NJ (US); Yamin Wang, Sandy Hook, CT (US); Jeremy L Baryza, Mountain View, CA (US); Philip Coish, New Haven, CT (US); William Bullock, Easton, CT (US); Roger Smith, Madison, CT (US); Jinshan Chen, Clinton, CT (US)

Correspondence Address: JEFFREY M. GREENMAN, BAYER PHARMACEUTICALS CORPORATION, 400 MORGAN LANE, WEST HAVEN, CT, 06516, US

(73) Assignee: Bayer Pharmaceuticals Corporation, West Haven, CT (US)

(21) Appl. No.: 10/499,786

(22) PCT Filed: Dec. 28, 2002
(86) PCT No.: PCT/US02/41634

Related U.S. Application Data
(60) Provisional application No. 60/344,149, filed on Dec. 28, 2001.

(51) Int. Cl. A61K 31/416; C07D 43/02; A61K 31/415

(52) U.S. Cl. 514/210.2; 514/217.09; 514/326; 514/406; 548/364.1; 548/374.1; 548/375.1; 546/211; 540/603

(57) ABSTRACT

Disclosed are pyrazolyl derivative compounds or purified stereoisomers or stereoisomer mixtures of said compounds and their salts or prodrug forms thereof which have structural formula (I) or (II): wherein the variables R, R₁, R₂, R₅ and R₆ are as defined in the specification. The compounds are useful for the treatment or prevention of diseases and/or behaviors involving the 5-HT_{2C} receptor.

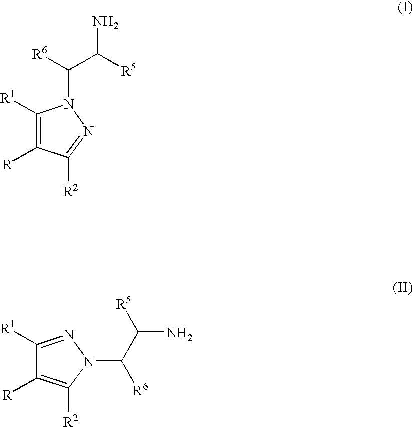

The present invention relates to:
(1) 1H-pyrazolyl derivative compounds or purified stereoisomers or stereoisomer mixtures of said compounds and salts or prodrug forms thereof;
(2) Pharmaceutical compositions comprising one or more of the compounds or purified stereoisomers or stereoisomer mixtures of the invention, or their salt or prodrug forms thereof, with a pharmaceutically acceptable ingredient;
(3) Methods of preparing the compounds of (1); and
(4) Methods of treating diseases associated with the 5-HT_{2C} receptor in mammals by administering an effective amount of (1) or (2) to a patient in need thereof.
   Description of the Compounds and Intermediates Thereof

The 1H-pyrazolyl derivative compounds or purified stereoisomers or stereoisomer mixtures of said compounds and their salts or prodrug forms thereof have the structural formulae: 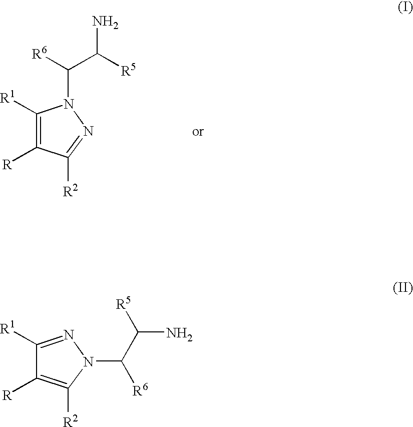
wherein:
R is selected from the group consisting of:
   (a) hydrogen,
   (b) (C₁-C₅)-alkyl optionally substituted with:
      (b1) (C₃-C₈)-cycloalkyl, or
      (b2) a four to eight membered saturated or unsaturated heterocyclic ring which contains one to four heteroatoms selected from the group consisting of nitrogen, oxygen and sulfur, wherein said heterocyclic ring contains at least one carbon atom and wherein said heterocyclic ring is optionally substituted with one to two —C(═O)R₉,
   (c) (C₁-C₅)-alkenyl optionally substituted with (C₁-C₅)-alkyl,
   (d) (C₁-C₅)-alkynyl optionally substituted with (C₁-C₅)-alkyl,
   (e) (C₆-C₁₀)-aryl optionally substituted with one to three substituents selected from the group consisting of:
      (e1) halogen,
      (e2) (C₁-C₅)-alkyl optionally substituted by halogen, and
      (e3) (C₁-C₅)-alkoxy,
   (f) (C₆-C₁₀)aryl-(C₁-C₅)-alkyl wherein the aryl is optionally substituted with one to three substituents selected from the group consisting of:
      (f1) halogen,
      (f2) nitro,
      (f3) (C₁-C₅)alkyl optionally substituted by halogen,
      (f4) (C₆-C₁₀)-aryl optionally substituted with one to three substituents selected from the group consisting of:
         (f4a) halogen,
         (f4b) (C₁-C₅)-alkyl optionally substituted with halogen, and
         (f4c) (C₁-C₅)-alkoxy,
      (f5) (C₆-C₁₀)-aryl-(C₁-C₅)-alkoxy,
      (f6) a four to eight membered saturated or unsaturated heterocyclic ring which contains one to four heteroatoms selected from the group consisting of nitrogen, oxygen and sulfur wherein said heterocyclic ring contains at least one carbon atom wherein said heterocyclic ring is optionally substituted with one to four substituents selected from the group consisting of:
         (f6a) oxo,
         (f6b) (C₁-C₅)-alkyl optionally substituted with (C₃-C₈)-spiro-cycloalkyl ring,
         (f6c) (C₆-C₁₀)-aryl,
         (f6d) (C₃-C₈)-spiro-cycloalkyl ring,
         (f6e) a bicyclo cycloalkyl ring wherein each ring is independently a five to six membered cycloalkyl ring,
         (f6f) a tricyclo cycloalkyl ring wherein each ring is independently a five to six membered cycloalkyl ring,
         (f6g) (C₁-C₅)-dialkyl,
      (f7) a fused bicyclo ring wherein one ring is a four to eight membered saturated or unsaturated heterocyclic ring which contains one to four heteroatoms selected from the group consisting of nitrogen, oxygen and sulfur, wherein said heterocyclic ring contains at least one carbon atoms and said heterocyclic ring is optionally substituted with one to two oxo substituents, and the other ring is a saturated or unsaturated three to eight membered cycloalkyl ring,
      (f8) NR³R⁴,
      (f9) NR¹⁸C(═O)—R¹⁴
      (f10) NR³S(═O)—R¹⁵, and
      (f11) C(═O)NR¹³C(═O)R¹⁶,
   (g) a fused bicyclo ring wherein both rings are saturated or unsaturated five to six membered cycloalkyl rings, and
   (h) a four to eight membered saturated or unsaturated heterocyclic ring which contains one to four heteroatoms selected from the group consisting of nitrogen, oxygen and sulfur, wherein said heterocyclic ring contains at least one carbon atom and wherein said heterocyclic ring is optionally substituted with one to four substituents selected from the group consisting of:
      (h1) C(═O)R⁹,
      (h2) C(═O)OR⁹,
      (h3) C(═O)NR¹⁰R¹¹,
      (h4) SO₂R¹²;
   (i) NR³R⁴;
   (j) NO₂, and
   (j) OR⁸;
R¹ and R² are independently selected from the group consisting of
   (a) hydrogen,
   (b) (C₁-C₅)-alkyl optionally substituted with halogen,
   (c) (C₂-C₅)-alkenyl optionally substituted with (C₁-C₅)-alkyl,
   (d) (C₂-C₅)-alkynyl optionally substituted with (C₁-C₅)-alkyl,
   (e) C(═O)R⁷,
   (f) C(═O)OR⁷, and
   (g) C(═O)NR³R⁴;
R³ and R⁴ are independently selected from the group consisting of
   (a) hydrogen,
   (b) (C₁-C₅)-alkyl optionally substituted with C₃-C₈-cycloalkyl,
   (c) (C₆-C₁₀)-aryl,
   (d) (C₆-C₁₀)-aryl-C₁-C₅-alkyl optionally substituted with one to three halogens,
   (e) C(═O)R⁹,
   (f) C(═O)OR⁹,
   (g) C(═O)NR¹⁰R¹¹,
   (h) (C₃-C₈)-cycloalkyl, and
   (i) —SO₂R¹²;
R⁵, R⁶ and R⁷ are independently selected from the group consisting of
   (a) hydrogen, and
   (b) (C₁-C₅)-alkyl;
R⁸ is selected from the group consisting of:
   (a) hydrogen,
   (b) (C₁-C₆)-alkyl optionally substituted with
      (b1) halogen,
      (b2) cyano, and
      (b3) a four to eight membered saturated or unsaturated heterocyclic ring which contains one to four heteroatoms selected from the group consisting of nitrogen, oxygen and sulfur, wherein said heterocyclic ring contains at least one carbon atom and wherein said heterocyclic ring is optionally substituted with nitro,
   (c) (C₂-C₅)-alkenyl optionally substituted with (C₁-C₅)alkyl,
   (d) (C₂-C₅)-alkynyl optionally substituted with (C₁-C₅)-alkyl,
   (e) (C₆-C₁₀)-aryl optionally substituted by one to three halogens,
   (f) (C₆-C₁₀)-aryl-C₁-C₅-alkyl wherein the (C₆-C₁₀)-aryl is optionally substituted with one to three substituents selected from the group consisting of cyano, halogen, (C₁-C₅)-alkoxy and phenyl,
   (g) (C₆-C₁₀)-aryloxy-(C₁-C₅)-alkyl,
   (h) (C₁-C₅)-alkyl-C(═O), and
   (i) (C₆-C₁₀)-aryl-C(═O);
R⁹ is selected from the group consisting of:
   (a) hydrogen,
   (b) (C₁-C₅)-alkyl optionally substituted with (C₃-C₈)-cycloalkyl,
   (c) (C₆-C₁₀)-aryl optionally substituted by one to three substituents selected from the group consisting of:
      (b1) halogen,
      (b2) (C₁-C₅)-alkyl,
      (b3) (C₁-C₅)-alkoxy, and
      (b4) (C₃-C₈)-cycloalkyl,
   (d) (C₆-C₁₀)-aryl-(C₁-C₅)-alkyl optionally substituted with halogen,
   (e) a four to eight membered saturated or unsaturated heterocyclic ring which contains one to four heteroatoms selected from the group consisting of nitrogen, oxygen and sulfur, wherein said heterocyclic ring contains at least one carbon atom, and
   (f) (C₃-C₈)-cycloalkyl;
R¹⁰ and R¹¹ are independently selected from the group consisting of:
   (a) hydrogen,
   (b) (C₁-C₅)-alkyl,
   (c) (C₆-C₁₀)-aryl optionally substituted with one to three substituents selected from the group consisting of:
      (c1) halogen,
      (c2) (C₁-C₅)-alkyl, and
      (c3) (C₁-C₅)-alkoxy,
   (d) (C₆-C₁₀)-aryl-(C₁-C₅)-alkyl wherein the (C₆-C₁₀)-aryl is optionally substituted with one to three substituents selected from the group consisting of:
      (d1) halogen,
      (d2) (C₁-C₅)-alkyl, and
      (d3) (C₁-C₅)-alkoxy, and
   (e) (C₃-C₈)-cycloalkyl;
R¹² is selected from the group consisting of
   (a) (C₁-C₅)-alkyl,
   (b) (C₆-C₁₀)-aryl optionally substituted with one to three substituents selected from the group consisting of:
      (b1) halogen,
      (b2) (C₁-C₅)-alkyl,
      (b3) (C₁-C₅)-alkoxy, and
   (c) C₆-C₁₀-aryl-(C₁-C₅)-alkyl wherein the C₆-C₁₀-aryl is optionally substituted with one to three substituents selected from the group consisting of:
      (c1) halogen,
      (c2) (C₁-C₅)-alkyl, and
      (c3) (C₁-C₅>alkoxy;
R¹³ is selected from the group consisting of hydrogen and (C₁-C₅)-alkyl;
R¹⁴ is selected from the group consisting of
   (a) (C₁-C₅{alkyl optionally substituted with substituents (C₁-C₅)-alkoxy, (C₃-C₈)-spiro-cycloalkyl, or (C₆-C₁₀)-aryl optionally substituted with one to three halogens,
   (b) (C₆-C₁₀)-aryl optionally substituted with one to three substituents selected from the group consisting of halogen, (C₁-C₅)-alkoxy, and (C₁-C₅)-alkyl optionally substituted with halogen,
   (c) (C₃-C₈)-cycloalkyl optionally substituted with one to three substituents selected from the group consisting of halogen, (C₁-C₅)-alkoxy, and (C₁-C₅)-alkyl optionally substituted with halogen, and
   (d) a bicyclo cycloalkyl ring wherein each ring is independently a five to six membered cycloalkyl ring,
   (e) a tricyclo cycloalkyl ring wherein each ring is independently a five to six membered cycloalkyl ring, and
   (f) a four to eight membered saturated or unsaturated heterocyclic ring which contains one to four heteroatoms selected from the group consisting of nitrogen, oxygen and sulfur, wherein said heterocyclic ring contains at least one carbon atom and wherein said ring is optionally substituted with one to three substituents selected from the group consisting of halogen, (C₁-C₅)-alkoxy, and (C₁-C₅)-alkyl optionally substituted with halogen;
R¹⁵ is selected from the group consisting of
   (a) hydrogen, and
   (b) (C₁-C₅)-alkyl;
R¹⁶ is selected from the group consisting of
   (a) hydrogen,
   (b) (C₁-C₅)-alkyl,
   (c) (C₃-C₈)-cycloalkyl, and
   (d) a bicyclo cycloalkyl ring wherein each ring is independently a five to six membered cycloalkyl ring, and
   (e) a tricyclo cycloalkyl ring wherein each ring is independently a five to six membered cycloalkyl ring;
      or a purified stereoisomer or stereoisomer mixture of said compound, or salt of said compound, stereoisomer or stereoisomer mixture.

### DETAILED DESCRIPTION

The preferred compounds of the invention have general formulae (I) and (II), and are further defined below. In the following description of these preferred compounds, the definitions for the various groups and variables represent the preferred definitions when they differ from those as broadly defined above, and are to be understood as independent of each other.

The preferred 1H-pyrazolyl derivative compounds or purified stereoisomers or stereoisomer mixtures of said compounds and their salts or prodrug forms thereof have the structural formulae: 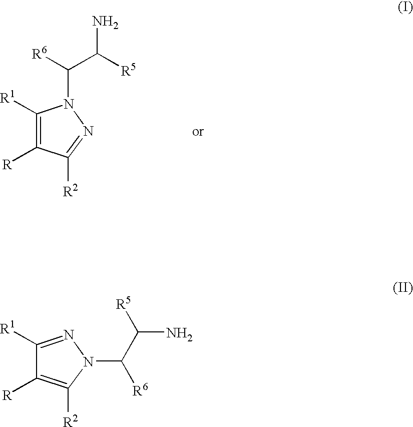
wherein:
R is selected from the group consisting of:
   (a) (C₁-C₅)-alkyl optionally substituted with:
      (a1) (C₃-C₈)-cycloalkyl, or
      (a2) a four to eight membered saturated or unsaturated heterocyclic ring which contains one to four heteroatoms selected from the group consisting of nitrogen, oxygen and sulfur, wherein said heterocyclic ring contains at least one carbon atom and wherein said heterocyclic ring is optionally substituted with one to two —C(═O)R⁹,
   (b) (C₁-C₅)-alkenyl optionally substituted with (C₁-C₅)-alkyl,
   (c) (C₁-C₅)-alkynyl optionally substituted with (C₁-C₅)-alkyl, and
   (d) (C₆-C₁₀)aryl-(C₁-C₅)-alkyl wherein the aryl is optionally substituted with one to three substituents selected from the group consisting of:
      (d1) halogen,
      (d2) nitro,
      (d3) (C₁-C₅)-alkyl optionally substituted by halogen,
      (d4) (C₆-C₁₀)-aryl optionally substituted with one to three substituents selected from the group consisting of:
         (d4a) halogen,
         (d4b) (C₁-C₅)-alkyl optionally substituted with halogen, and
         (d4c) (C₁-C₅)-alkoxy,
      (d5) (C₆-C₁₀)-aryl-(C₁-C₅)-alkoxy,
      (d6) a four to eight membered saturated or unsaturated heterocyclic ring which contains one to four heteroatoms selected from the group consisting of nitrogen, oxygen and sulfur wherein said heterocyclic ring contains at least one carbon atom wherein said heterocyclic ring is optionally substituted with one to four substituents selected from the group consisting of:
         (d6a) oxo,
         (d6b) (C₁-C₅)-alkyl optionally substituted with (C₃-C₈)-spiro-cycloalkyl ring,
         (d6c) (C₆-C₁₀)-aryl,
         (d6d) (C₃C₈)-spiro-cycloalkyl ring,
         (d6e) a bicyclo cycloalkyl ring wherein each ring is independently a five to six membered cycloalkyl ring,
         (d6f) a tricyclo cycloalkyl ring wherein each ring is independently a five to six membered cycloalkyl ring, and
         (d6g) (C₁-C₅)-dialkyl,
      (d7) a fused bicyclo ring wherein one ring is a four to eight membered saturated or unsaturated heterocyclic ring which contains one to four heteroatoms selected from the group consisting of nitrogen, oxygen and sulfur, wherein said heterocyclic ring contains at least one carbon atoms and said heterocyclic ring is optionally substituted with one to two oxo substituents, and the other ring is a saturated or unsaturated three to eight membered cycloalkyl ring,
      (d8) NR³R⁴,
      (d9) NR¹³C(═O)—R¹⁴,
      (d10) NR¹³S(═O)—R¹⁵, and
      (d11) C(═O)NR¹³C(═O)R¹⁶
R¹ and R² are independently selected from the group consisting of
   (a) hydrogen,
   (b) (C₁-C₅)-alkyl optionally substituted with halogen,
   (c) (C₂-C₅)-alkenyl optionally substituted with (C₁-C₅)-alkyl, and
   (d) (C₂-C₅)-alkynyl optionally substituted with (C₁-C₅)-alkyl;
R³ and R⁴ are independently selected from the group consisting of
   (a) hydrogen,
   (b) (C₁-C₅)-alkyl optionally substituted with C₃-C₈-cycloalkyl,
   (c) (C₆-C₁₀)-aryl,
   (d) (C₆-C₁₀)-aryl-C₁-C₅-alkyl optionally substituted with one to three halogens,
   (e) C(═O)R⁹,
   (f) C(═O)NR¹⁰R¹¹,
   (g) (C₃-C₈)cycloalkyl, and
   (h) —SO₂R¹²;
R⁵ and R⁶ are independently selected from the group consisting of
   (a) hydrogen, and
   (b) (C₁-C₅)-alkyl;
R⁹ is selected from the group consisting of:
   (a) hydrogen,
   (b) (C₁-C₅)-alkyl optionally substituted with (C₃-C₈)-cycloalkyl,
   (c) (C₆-C₁₀)-aryl optionally substituted by one to three substituents selected from the group consisting of:
      (c1) halogen.
      (c2) (C₁-C₅)-alkyl,
      (c3) (C₁-C₅)-alkoxy, and
      (c4) (C₃-C₈)-cycloalkyl,
   (d) (C₆-C₁₀)aryl-(C₁-C₅)-alkyl optionally substituted with halogen,
   (e) a four to eight membered saturated or unsaturated heterocyclic ring which contains one to four heteroatoms selected from the group consisting of nitrogen, oxygen and sulfur, wherein said heterocyclic ring contains at least one carbon atom, and
   (f) (C₃-C₈)-cycloalkyl;
R¹⁰ and R¹¹ are independently selected from the group consisting of:
   (a) hydrogen,
   (b) (C₁-C₅)-alkyl,
   (c) (C₆-C₁₀)-aryl optionally substituted with one to three substituents selected from the group consisting of:
      (c1) halogen,
      (c2) (C₁-C₅)-alkyl, and
      (c3) (C₁-C₅)-alkoxy, and
   (d) (C₃-C₈)-cycloalkyl;
R¹² is (C₁-C₅)-alkyl;
R¹³ is selected from the group consisting of hydrogen and (C₁-C₅)-alkyl;
R¹⁴ is selected from the group consisting of
   (a) (C₁-C₅)alkyl optionally substituted with substituents (C₁-C₅)-alkoxy, (C₃-C₈) spiro-cycloalkyl, or (C₆-C₁₀)-aryl optionally substituted with one to three halogens,
   (b) (C₆-C₁₀)-aryl optionally substituted with one to three substituents selected from the group consisting of halogen, (C₁-C₅)-alkoxy, and (C₁-C₅)-alkyl optionally substituted with halogen, and
   (c) (C₃-C₈)-cycloalkyl optionally substituted with one to three substituents selected from the group consisting of halogen, (C₁-C₅)-alkoxy, and (C₁-C₅)-alkyl optionally substituted with halogen;
R¹⁵ is selected from the group consisting of
   (a) hydrogen, and
   (b) (C₁-C₅)-alkyl;
R¹⁶ is selected from the group consisting of
   (a) hydrogen,
   (b) (C₁-C₅)-alkyl, and
   (c) (C₃-C₈)-cycloalkyl;
      or a purified stereoisomer or stereoisomer mixture of said compound, or salt of said compound, stereoisomer or stereoisomer mixture.

The more preferred compounds of the invention have general formulae (I) and (II), and are further defined below. In the following description of these more preferred compounds, the definitions for the various groups and variables represent the preferred definitions when they differ from those as broadly defined above, and are to be understood as independent of each other.

The more preferred 1H-pyrazolyl derivative compounds or purified stereoisomers or stereoisomer mixtures of said compounds and their salts or prodrug forms thereof have the structural formulae: 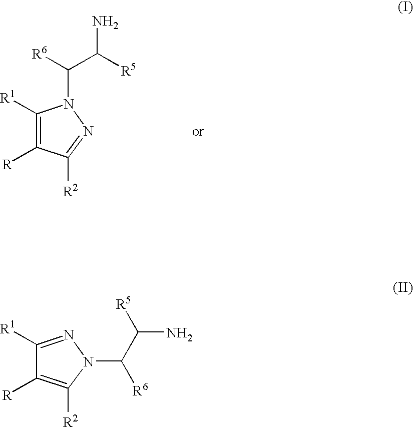
wherein:
R is selected from the group consisting of:
   (a) (C₆-C₁₀)-aryl-(C₁-C₅)-alkyl wherein the aryl is optionally substituted with one to three substituents selected from the group consisting of:
      (a1) halogen,
      (a2) nitro,
      (a3) (C₁-C₅)-alkyl optionally substituted by halogen,
      (a4) (C₆-C₁₀)-aryl optionally substituted with one to three substituents selected from the group consisting of:
         (a4a) halogen,
         (a4b) (C₁-C₅)-alkyl optionally substituted with halogen, and
         (a4c) (C₁-C₅)-alkoxy,
      (a5) (C₆-C₁₀)-aryl-(C₁-C₅)-alkoxy,
      (a6) a four to eight membered saturated or unsaturated heterocyclic ring which contains one to four heteroatoms selected from the group consisting of nitrogen, oxygen and sulfur wherein said heterocyclic ring contains at least one carbon atom wherein said heterocyclic ring is optionally substituted with one to four substituents selected from the group consisting of:
         (a6a) oxo,
         (a6b) (C₁-C₅)-alkyl optionally substituted with (C₃-C₈)-spiro-cycloalkyl ring,
         (a6c) (C₆-C₁₀)-aryl,
         (a6d) (C₃-C₈)-spiro-cycloalkyl ring,
         (a6e) a bicyclo cycloalkyl ring wherein each ring is independently a five to six membered cycloalkyl ring,
         (a6f) a tricyclo cycloalkyl ring wherein each ring is independently a five to six membered cycloalkyl ring, and
         (a6g) (C₁-C₅)-dialkyl,
      (a7) a fused bicyclo ring wherein one ring is a four to eight membered saturated or unsaturated heterocyclic ring which contains one to four heteroatoms selected from the group consisting of nitrogen, oxygen and sulfur, wherein said heterocyclic ring contains at least one carbon atoms and said heterocyclic ring is optionally substituted with one to two oxo substituents, and the other ring is a saturated or unsaturated three to eight membered cycloalkyl ring,
      (a8) NR³R⁴,
      (a9) NR¹³C(═O)—R¹⁴,
      (a10) NR¹³S(═O)—R¹⁵, and
      (a11) C(═O)NR¹³C(═O)R¹⁶; R¹ and R² are independently selected from the group consisting of
   (a) hydrogen, and
   (b) (C₁-C₅)-alkyl optionally substituted with halogen;
R³ and R⁴ are independently selected from the group consisting of
   (a) hydrogen,
   (b) (C₁-C₅)-alkyl optionally substituted with C₃-C₈-cycloalkyl,
   (c) C(═O)R⁹,
   (d) C(═O)NR¹⁰R¹¹,
   (e) (C₃-C₈)-cycloalkyl, and
   (f) —SO₂R¹²;
R⁵ and R⁶ are independently selected from the group consisting of
   (a) hydrogen, and
   (b) (C₁-C₅)-alkyl,
R⁹ is selected from the group consisting of:
   (a) hydrogen,
   (b) (C₁-C₅)-alkyl optionally substituted with (C₃-C₈)-cycloalkyl,
   (c) (C₆-C₁₀)-aryl optionally substituted by one to three substituents selected from the group consisting of:
      (c1) halogen,
      (c2) (C₁-C₅)-alkyl,
      (c3) (C₁-C₅)-alkoxy, and
      (c4) (C₃-C₈)-cycloalkyl,
   (d) (C₆-C₁₀)-aryl-(C₁-C₅)-alkyl optionally substituted with halogen,
   (e) a four to eight membered saturated or unsaturated heterocyclic ring which contains one to four heteroatoms selected from the group consisting of nitrogen, oxygen and sulfur, wherein said heterocyclic ring contains at least one carbon atom, and
   (f) (C₃-C₈)-cycloalkyl;
R¹⁰ and R¹¹ are independently selected from the group consisting of:
   (a) hydrogen,
   (b) (C₁-C₅)-alkyl,
   (c) (C₆-C₁₀)-aryl optionally substituted with one to three substituents selected from the group consisting of:
      (c1) halogen,
      (c2) (C₁-C₅)-alkyl, and
      (c3) (C₁-C₅)-alkoxy, and
   (d) (C₃-C₈)-cycloalkyl;
R¹² is (C₁-C₅)-alkyl;
R¹³ is selected from the group consisting of hydrogen and (C₁-C₅)-alkyl;
R¹⁴ is selected from the group consisting of
   (a) (C₁-C₅)alkyl optionally substituted with substituents (C₁-C₅)-alkoxy, (C₃-C₈)-spiro-cycloalkyl, or (C₆-C₁₀)-aryl optionally substituted with one to three halogens,
   (b) (C₆-C₁₀)-aryl optionally substituted with one to three substituents selected from the group consisting of halogen, (C₁-C₅)-alkoxy, and (C₁-C₅)-alkyl optionally substituted with halogen, and
   (c) (C₃-C₈)-cycloalkyl optionally substituted with one to three substituents selected from the group consisting of halogen, (C₁-C₅)-alkoxy, and (C₁-C₅)-alkyl optionally substituted with halogen; and
R¹⁵ is selected from the group consisting of
   (a) hydrogen, and
   (b) (C₁-C₅)-alkyl;
R¹⁶ is selected from the group consisting of
   (a) hydrogen,
   (b) (C₁-C₅)-alkyl, and
   (c) (C₃-C₈)-cycloalkyl;
      or a purified stereoisomer or stereoisomer mixture of said compound, or salt of said compound, stereoisomer or stereoisomer mixture.

The compounds of the present invention may contain asymmetric centers on the molecule, depending upon the nature of the various substituents. Each such asymmetric center will produce two optical isomers. In certain instances, asymmetry may also be present due to restricted rotation about a central bond joining the two aromatic rings of the specified compounds. It is intended that all isomers, either by nature of asymmetric centers or by restricted rotation as described above, as separated, pure or partially purified isomers or racemic mixtures thereof, be included within the scope of the invention.

In cases in which the compounds have unsaturated carbon-carbon double bonds, both the cis (Z) and trans (E) isomers are within the scope of this invention.

In cases where the compounds may exist in tautomeric forms, each tautomeric form is contemplated as being encompassed by the scope of the invention whether existing in equilibrium with its corresponding tautomeric form or whether set in that form due through chemical derivatization.

Pharmaceutically acceptable salts of these compounds as well as commonly used prodrugs of these compounds are also within the scope of the invention.

Salts are especially the pharmaceutically acceptable salts of compounds of formulas (I) or (II) such as, for example, organic or inorganic acid addition salts of compounds of formulas (I) or (II). Suitable inorganic acids include but are not limited to halogen acids (such as hydrochloric acid), sulfuric acid, or phosphoric acid. Suitable organic acids include but are not limited to carboxylic, phosphonic, sulfonic, or sulfamic acids, with examples including acetic acid, trifluoroacetic acid, propionic acid, octanoic acid, decanoic acid, dodecanoic acid, glycolic acid, lactic acid, 2- or 3-hydroxybutyric acid, γ-aminobutyric acid (GABA), gluconic acid, glucosemonocarboxylic acid, benzoic acid, salicylic acid, phenylacetic acid, mandelic acid, methanesulfonic acid, trifluoromethanesulfonic acid, fumaric acid, oxalic acid, succinic acid, adipic acid, pimelic acid, suberic acid, azelaic acid, malic acid, tartaric acid, citric acid, glucaric acid, galactaric acid, amino acids (such as glutamic acid, aspartic acid, N-methylglycine, acetytaminoacetic acid, N-acetylasparagine or N-acetylcysteine), pyruvic acid, acetoacetic acid, phosphoserine, and 2- or 3-glycerophosphoric acid.

In addition, pharmaceutically acceptable salts include acid salts of inorganic bases, such as salts containing alkaline cations (e.g., Li⁺ Na⁺ or K⁺), alkaline earth cations (e.g., Mg⁺², Ca⁺² or Ba⁺²), the ammonium cation, as well as acid salts of organic bases, including aliphatic and aromatic substituted ammonium, and quaternary ammonium cations such as those arising from protonation or peralkylation of triethylamine, N,N-diethylamine, N,N-dicyclohexylamine, pyridine, N,N-dimethylaminopyridine (DMAP), 1,4-diazabicyclo[2.2.2]octane (DABCO), 1,5-diazabicyclo[4.3.0]non-5-ene (DBN) and 1,8-diazabicyclo[5.4.0]undec-7-ene (DBU).

Prodrugs are considered to be any covalently bonded carriers which release the active parent compound of formula (I) or (II) in vivo. Formation of prodrugs is well known in the art in order to enhance the properties of the parent compound; such properties include solubility, absorption, biostability and release time (see “Pharmaceutical Dosage Form and Drug Delivery Systems” (Sixth Edition), edited by Ansel et al., publ. by Williams & Wilkins, pgs. 27-29, (1995) which is hereby incorporated by reference).

Commonly used prodrugs of the disclosed compounds of formula (I) and (II) are designed to take advantage of the major drug biotransformation reactions and are also to be considered within the scope of the invention. Major drug biotransformation reactions include N-dealkylation, O-dealkylation, aliphatic hydroxylation, aromatic hydroxylation, N-oxidation, S-oxidation, deamination, hydrolysis reactions, glucuronidation, sulfation and acetylation (see Goodman and Gilman's The Pharmacological Basis of Therapeutics (Tenth Edition), editor Hardman et al., publ. by McGraw-Hill, pages 12-18, (2001), which is hereby incorporated by reference).

Definitions

The term “halogen” or “halo” as it appears in the specification and claims refers to fluorine, chlorine, bromine, and iodine substituents for the purposes of this invention. When halogen is a possible substituent on an alkyl group, the alkyl may be fully substituted, up to perhalo.

The term “dialkyl” as it appears in the specification and claims refers to double substitution with an alkyl substituent (see example below for illustration):

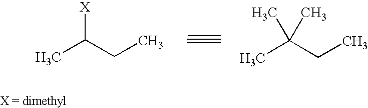

The term “fused bicyclo ring” as it appears in the specification and claims refers to a substituent which is a two ring structure which share two carbon atoms. The bonding between the fused bicyclo ring and the compound and/or atom to which it is attached can be through either of the two rings.

The term “spiro” ring as it appears in the specification and claims refers to a two ring system having one atom in common (e.g. a spiro ring attached to a phenyl group means that the spiro ring shared a carbon with the phenyl group).

Description of the Compositions

The invention also includes pharmaceutical compositions comprising one or more of the compounds of Formula (I) or (II), or a purified stereoisomer or stereoisomer mixture or their salt or prodrugs form thereof, with a pharmaceutically acceptable ingredient.

The invention also relates to pharmaceutical compositions containing a therapeutically effective amount of the compounds of Formula (I) and (II), or a purified stereoisomer or stereoisomer mixture or their salt or prodrug form thereof, and their use in combination with other drugs or therapies for the treatment of diseases and/or behaviors associated with the 5-HT_{2C} receptor.

The pharmaceutical compositions are prepared so that they may be administered orally, dermally, parenterally, nasally, ophthalmically, otically, sublingually, rectally or vaginally. Dermal administration includes topical application or transdermal administration. Parenteral administration includes intravenous, intraarticular, intramuscular, and subcutaneous injections, as well as use of infusion techniques. One or more compounds of the invention may be present in association with one or more non-toxic pharmaceutically acceptable ingredients and optionally, other active anti-proliferative agents, to form the pharmaceutical composition. These compositions can be prepared by applying known techniques in the art such as those taught in Remington's Pharmaceutical Sciences (Fourteenth Edition), Managing Editor, John E. Hoover, Mack Publishing Co., (1970) or Pharmaceutical Dosage Form and Drug Delivery Systems (Sixth Edition), edited by Ansel et al., publ. by Williams & Wilkins, (1995), each of which is hereby incorporated by reference.

Commonly used pharmaceutical ingredients which can be used as appropriate to formulate the composition for its intended route of administration include:
acidifying agents (examples include but are not limited to acetic acid, citric acid, fumaric acid, hydrochloric acid, nitric acid);
alkalinizing agents (examples include but are not limited to ammonia solution, ammonium carbonate, diethanolamine, monoethanolamine, potassium hydroxide, sodium borate, sodium carbonate, sodium hydroxide, triethanolamine, trolamine);
adsorbents (examples include but are not limited to powdered cellulose and activated charcoal);
aerosol propellants (examples include but are not limited to carbon dioxide, CCl₂F₂, F₂ClC-CClF₂ and CClF₃)
air displacement agents (examples include but are not limited to nitrogen and argon);
antifungal preservatives (examples include but are not limited to benzoic acid, butylparaben, ethylparaben, methylparaben, propylparaben, sodium benzoate);
antimicrobial preservatives (examples include but are not limited to benzalkonium chloride, benzethonium chloride, benzyl alcohol, cetylpyridinium chloride, chlorobutanol, phenyl, phenylethyl alcohol, phenylmercuric nitrate and thimerosal);
antioxidants (examples include but are not limited to ascorbic acid, ascorbyl palmitate, butylated hydroxyanisole, butylated hydroxytoluene, hypophosphorus acid, monothioglycerol, propyl gallate, sodium ascorbate, sodium bisulfite, sodium formaldehyde sulfoxylate, sodium metabisulfite);
binding materials (examples include but are not limited to block polymers, natural and synthetic rubber, polyacrylates, polyurethanes, silicones and styrene-butadiene copolymers);
buffering agents (examples include but are not limited to potassium metaphosphate, potassium phosphate monobasic, sodium acetate, sodium citrate anhydrous and sodium citrate dihydrate)
carrying agents (examples include but are not limited to acacia syrup, aromatic syrup, aromatic elixir, cherry syrup, cocoa syrup, orange syrup, syrup, corn oil, mineral oil, peanut oil, sesame oil, bacteriostatic sodium chloride injection and bacteriostatic water for injection)
chelating agents (examples include but are not limited to edetate disodium and edetic acid)
colorants (examples include but are not limited to FD&C Red No. 3, FD&C Red No. 20, FD&C Yellow No. 6, FD&C Blue No. 2, D&C Green No. 5, D&C Orange No. 5, D&C Red No. 8, caramel and ferric oxide red);
clarifying agents (examples include but are not limited to bentonite);
emulsifying agents (examples include but are not limited to acacia, cetomacrogol, cetyl alcohol, glyceryl monostearate, lecithin, sorbitan monooleate, polyethylene 50 stearate);
encapsulating agents (examples include but are not limited to gelatin and cellulose acetate phthalate)
flavorants (examples include but are not limited to anise oil, cinnamon oil, cocoa, menthol, orange oil, peppermint oil and vanillin);
humectants (examples include but are not limited to glycerin, propylene glycol and sorbitol);
levigating agents (examples include but are not limited to mineral oil and glycerin);
oils (examples include but are not limited to arachis oil, mineral oil, olive oil, peanut oil, sesame oil and vegetable oil);
ointment bases (examples include but are not limited to lanolin, hydrophilic ointment, polyethylene glycol ointment, petrolatum, hydrophilic petrolatum, white ointment, yellow ointment, and rose water ointment);
penetration enhancers (transdermal delivery) (examples include but are not limited to monohydroxy or polyhydroxy alcohols, saturated or unsaturated fatty alcohols, saturated or unsaturated fatty esters, saturated or unsaturated dicarboxylic acids, essential oils, phosphatidyl derivatives, cephalin, terpenes, amides, ethers, ketones and ureas)
plasticizers (examples include but are not limited to diethyl phthalate and glycerin);
solvents (examples include but are not limited to alcohol, corn oil, cottonseed oil, glycerin, isopropyl alcohol, mineral oil, oleic acid, peanut oil, purified water, water for injection, sterile water for injection and sterile water for irrigation);
stiffening agents (examples include but are not limited to cetyl alcohol, cetyl esters wax, microcrystalline wax, paraffin, stearyl alcohol, white wax and yellow wax);
suppository bases (examples include but are not limited to cocoa butter and polyethylene glycols (mixtures));
surfactants (examples include but are not limited to benzalkonium chloride, nonoxynol 10, oxtoxynol 9, polysorbate 80, sodium lauryl sulfate and sorbitan monopalmitate);
suspending agents (examples include but are not limited to agar, bentonite, carbomers, carboxymethylcellulose sodium, hydroxyethyl cellulose, hydroxypropyl cellulose, hydroxypropyl methylcellulose, kaolin, methylcellulose, tragacanth and veegum);
sweetening agents (examples include but are not limited to aspartame, dextrose, glycerin, mannitol, propylene glycol, saccharin sodium, sorbitol and sucrose);
tablet anti-adherents (examples include but are not limited to magnesium stearate and talc);
tablet binders (examples include but are not limited to acacia, alginic acid, carboxymethylcellulose sodium, compressible sugar, ethylcellulose, gelatin, liquid glucose, methylcellulose, povidone and pregelatinized starch);
tablet and capsule diluents (examples include but are not limited to dibasic calcium phosphate, kaolin, lactose, mannitol, microcrystalline cellulose, powdered cellulose, precipitated calcium carbonate, sodium carbonate, sodium phosphate, sorbitol and starch);
tablet coating agents (examples include but are not limited to liquid glucose, hydroxyethyl cellulose, hydroxypropyl cellulose, hydroxypropyl methylcellulose, methylcellulose, ethylcellulose, cellulose acetate phthalate and shellac);
tablet direct compression excipients (examples include but are not limited to dibasic calcium phosphate);
tablet disintegrants (examples include but are not limited to alginic acid, carboxymethylcellulose calcium, microcrystalline cellulose, polacrillin potassium, sodium alginate, sodium starch glycollate and starch);
tablet glidants (examples include but are not limited to colloidal silica, corn starch and talc);
tablet lubricants (examples include but are not limited to calcium stearate, magnesium stearate, mineral oil, stearic acid and zinc stearate);
tablet/capsule opaquants (examples include but are not limited to titanium dioxide);
tablet polishing agents (examples include but are not limited to carnuba wax and white wax);
thickening agents (examples include but are not limited to beeswax, cetyl alcohol and paraffin);
tonicity agents (examples include but are not limited to dextrose and sodium chloride);
viscosity increasing agents (examples include but are not limited to alginic acid, bentonite, carbomers, carboxymethylcellulose sodium, methylcellulose, povidone, sodium alginate and tragacanth); and
wetting agents (examples include but are not limited to heptadecaethylene oxycetanol, lecithins, polyethylene sorbitol monooleate, polyoxyethylene sorbitol monooleate, polyoxyethylene stearate,).

Depending on the route of administration, the compositions can take the form of aerosols, capsules, creams, elixirs, emulsions, foams, gels, granules, inhalants, lotions, magmas, ointments, peroral solids, powders, sprays, syrups, suppositories, suspensions, tablets and tinctures.

Optional additional agents which can be added to the composition include but are not limited to compounds which are known to treat obesity and obesity related disorder such as diabetes, abnormal feeding behavior, eating disorders (such as bulimia nervosa and anorexia nervosa) and premenstrual tension.

Examples of agents for treating obesity include appetite suppressants such as benzphetamine, diethylpropion, Mazindol, phendimetrazine and phentennine.

Examples of agents for treating diabetes include insulin for insulin-dependent diabetes (IDDM) and sulfonylurea compounds for non-insulin dependent diabetes (NIDDM). Examples of sulfonylureas include tolbutamide, chlorpropamide, tolazamide, acetohexamide, glycburide, glipizide and gliclazide.

It had previously been disclosed that psychosomatic disorders such as bulimia nervosa may respond at least partly to treatment with antidepressants such as tricyclic monoamine oxidase (MAO) inhibitors and serotonin reuptake inhibitors (see Goodman and Gilman's The Pharmacological Basis of Therapeutics (Tenth Edition), editor Hardman et al., publ. by McGraw-Hill, page 469, (2001), the contents of which is hereby incorporated by reference. Likewise it would be expected that these agents (e.g. fluoxetine) in combination with the applicants described compounds would have similar effects.

For all regimens of use disclosed herein for compounds of formulas (I) or (II), the daily oral dosage regimen will preferably be from 0.01 to 200 mg/kg of total body weight. The daily dosage for administration by injection, including intravenous, intramuscular, subcutaneous and parenteral injections, and use of infusion techniques will preferably be from 0.01 to 200 mg/kg of total body weight. The daily rectal dosage regimen will preferably be from 0.01 to 200 mg/kg of total body weight. The daily vaginal dosage regimen will preferably be from 0.01 to 200 mg/kg of total body weight. The daily topical dosage regimen will preferably be from 0.1 to 200 mg administered between one to four times daily. The transdermal concentration will preferably be that required to maintain a daily dose of from 0.01 to 200 mg/kg. The daily inhalation dosage regimen will preferably be from 0.01 to 100 mg/kg of total body weight.

It will be appreciated by those skilled in the art that the particular method of administration will depend on a variety of factors, all of which are considered routinely when administering therapeutics. It will also be understood, however, that the specific dose level for any given patient will depend upon a variety of factors, including, but not limited to the activity of the specific compound employed, the age of the patient, the body weight of the patient; the general health of the patient, the gender of the patient, the diet of the patient, time of administration, route of administration, rate of excretion, drug combinations, and the severity of the condition undergoing therapy. It will be further appreciated by one skilled in the art that the optimal course of treatment, i.e., the mode of treatment and the daily number of doses of a compound of formulas (I) or (II) or a pharmaceutically acceptable salt thereof given for a defined number of days, can be ascertained by those skilled in the art using conventional treatment tests.

Description of Preparative Methods

Abbreviations and Acronyms

| | |
|---|---|
| Boc | tert-butoxycarbonyl |
| Cbz | carbobenzyloxy |
| DMF | dimethylformamide |
| ELSD | evaporative light scattering detector |
| ESLC | electrospray liquid chromatography |
| ES-MS | electrospray - mass spectroscopy |
| Et₂O | diethyl ether |
| Ms | mesyl |
| NBS | N-bromosuccinimide |
| rt | room temperature |
| Ts | tosyl |

Compounds of Formula 1 and 11 where R is bonded to the pyrazole ring with a carbon-carbon bond, may generally be prepared by the methods illustrated in Reaction Scheme I below. The bromo pyrazole starting materials 2 are either known in the literature or are generally prepared by condensation of hydrazine with a 1,3 diketone of formula R₁C(═O)CH₂C(═O)R₂, followed by halogenation under standard conditions, e.g., bromine/acetic acid or NBS. The conversion of 2 to 3 is accomplished by a Palladium-catalyzed coupling with a boronic acid or ester as shown. The catalyst may be Pd(dppf)Cl₂ and the like. N-substitution of 3 may be accomplished by its treatment with a substituted amine, 4, containing a leaving group X¹, where X¹ may be Br, I, Cl, methanesulfonate, tosylate or the like. The amine may be first protected if required, as for example the N-BOC derivative, and deprotected following the N-substitution reaction.

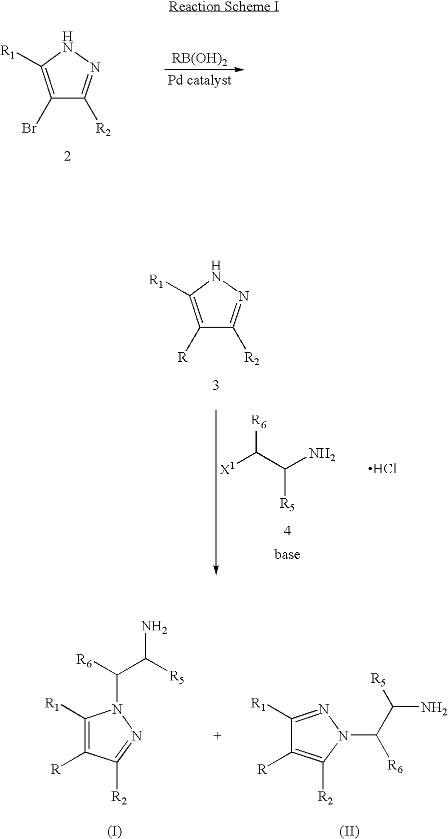

Compounds of Formula I and II where R represents a R₈O— or a R₃R₄N group, may generally be prepared by the methods illustrated in Reaction Scheme II below. 3-Substituted diketones of formula 7, in which R is R₈O— or R₃R⁴N may be converted to substituted or unsubstituted pyrazoles directly. The preparation of 7, if not commercially available, may be accomplished by elaboration of unsubstituted ketones 6 by straightforward means such as halogenation-substitution, or nitration-reduction-substitution. Protection/deprotection steps may be incorporated as required. It is understood that the amino of the —CH(R₅)CH(R⁶)—NH₂ portion of the compound of formula (I) and (II) may be protected and deprotected (e.g., BocNH—CH₂CH₂NHNH₂) as needed in order to carry out the Schemes below.

HPLC-electrospray mass spectra (HPLC ES-MS) were obtained using a Hewlett-Packard 1100 HPLC equipped with a quaternary pump, a variable wavelength detector, a YMC Pro C18 2.0 mm×23 mm column, and a Finnigan LCQ ion trap mass spectrometer with electrospray ionization. Gradient elution from 90% A to 95% B over 4 minutes was used on the HPLC. Buffer A was 98% water, 2% Acetonitrile and 0.02% TFA. Buffer B was 98% Acetonitrile, 2% water and 0.018% TFA. Spectra were scanned from 140-1200 amu using a variable ion time according to the number of ions in the source.

Unsubstituted pyrazoles of formula Ia may be converted to a mixture of formulas (I) and (II) compounds by action of an alkylating agent and a base.

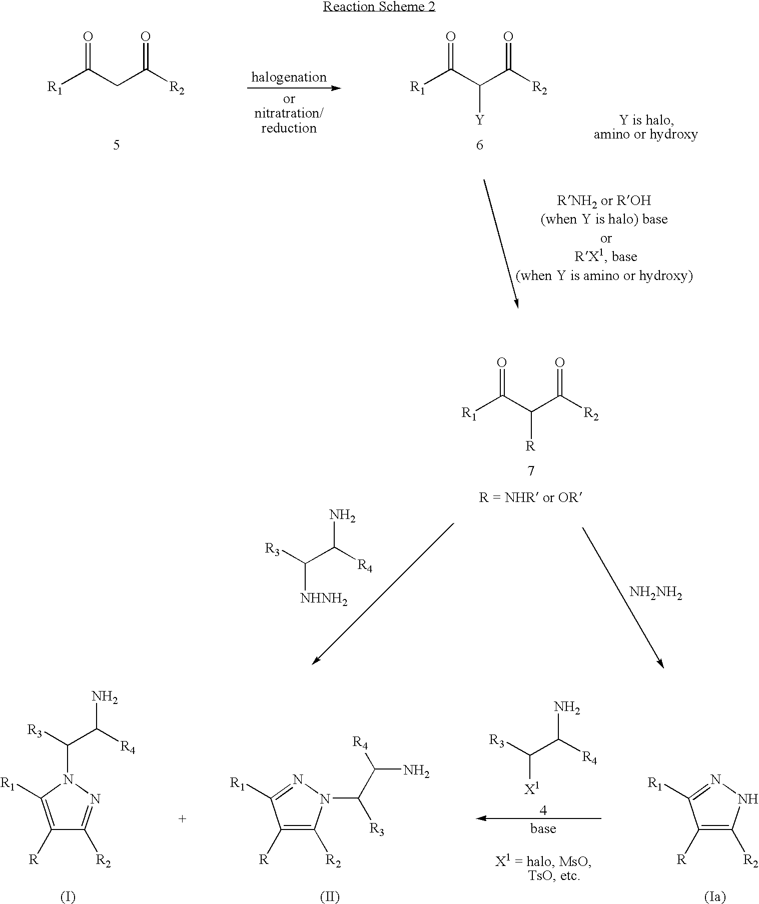

### EXAMPLE 1

### Preparation of 4-Phenyl-3,5-dimethylpyrazole

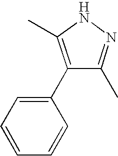

A mixture of 4-bromo-3,5-dimethylpyrazole (0.55 g, 3.1423 mmol), phenyl boronic acid (0.5 g, 4.0855 mmol), PdCl₂(PPh₃)₂ (0.044 g, 0.0628 mmol) and sodium carbonate (2.11 mL, 2N) in toluene (10 mL) was heated at 90° C. for 18 h and cooled to room temperature. Some ice was added and the mixture extracted with dichloromethane (3×30 mL) and dried over MgSO₄ and concentrated. The product was isolated by column chromatography (50% hexane in EtOAc) to give a cream colored solid (0.22 g, 41%). R_{f}=0.16 (50% hexane in EtOAc), GC/MS 172 (M⁺, 100%), ¹H NMR (300 MHz, CDCl₃) δ 7.46-7.39 (m, 2H), 7.33-7.28 (m, 3H), 2.34 (s, 6H).

### EXAMPLE 2

### Preparation of 4-Phenyl-3,5-dimethyl-pyrazol-1-yl)ethylamine

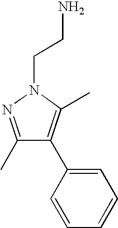

To a suspension of 4-phenyl-3,5-dimethylpyrazole (0.65 g, 3.774 mmol) in acetonitrile (2.4 mL) was added sodium hydroxide (0.6 g, 15.096 mmol). The mixture was stirred under argon for 30 min at room temperature. 2-Chloroethylamine hydrochloride (0.66 g, 5.661 mol) was added, followed by tetrabutylammonium hydrogen sulfate (0.051 g, 0.1509 mmol), the reaction mixture was stirred at reflux for 1.5 h and diluted with ethyl acetate (100 mL), dried over Na₂SO₄, filtered through a bed of Celite. The filtrate was concentrated. The residue was dissolved in ethyl acetate (20 mL) and passed through a silica gel plug, using ethyl acetate as the initial eluant, then 5% methanol in ethyl acetate and finally 10% methanol in ethyl acetate. The eluants were concentrated to give 0.604 g, 74% of product. R_{f}=0.10 (5% MeOH in EtOAc), GC/MS; 215 (M⁺), ¹H NMR (300 MHz, CDCl₃) δ 7.43-7.38 (m, 2H), 7.30-7.23 (m, 3H), 4.10 (t, 2H), 3.16 (t, 2H), 2.26 (s, 3H), 2.25 (s, 3H).

### EXAMPLE 3

### Preparation of 4-Phenyl-3,5-dimethyl-pyrazol-1-yl)ethylamine Hydrochloride

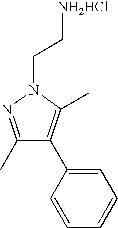

A solution of Example 2 (0.23 g, 1.0683 mmol) in ether (10 mL) was treated with HCl in ether (4.2 mL, 1 M). The mixture was stirred for 30 min and concentrated. The residue was washed twice with ether (15 mL) and dried under vacuum to give a cream colored solid (0.4 g, 100%). Mp 244-247° C., ¹H NMR (300 MHz, DMSO) δ 7.44-7.39 (m, 2H), 7.30-7.23 (m, 3H), 4.25 (t, 2H), 3.25 (m, 2H), 2.23 (s, 3H), 2.15 (s, 3H).

### EXAMPLE 4

### Preparation of 2-(4-Bromo-3,5-dimethyl-pyrazol-1-yl)ethylamine

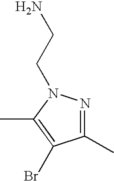

To a suspension of 4-bromo-3,5-dimethylpyrazole (5 g, 0.0286 mol) in acetonitrile (18 mL) was added sodium hydroxide (4.579, 0.1143 mol). The mixture was stirred under argon for 30 min at room temperature. 2-chloroethylamine hydrochloride (4.97 g, 0.0429 mol) was added, followed by tetrabutylammonium hydrogen sulfate (0.39 g, 0.0011 mol), the reaction mixture was stirred at reflux for 3 h and diluted with ethyl acetate (20 mL). The mixture was filtered and the filtrate concentrated. The residue was dissolved in ethyl acetate (35 mL), dried over Na₂SO₄, and filtered through a silica gel plug, using ethyl acetate as the initial eluant, then 5% methanol in ethyl acetate and finally 10% methanol in ethyl acetate. The eluants were concentrated to give 5 g, 80% of product. R_{f}=0.08 (5% MeOH in EtOAc), GC/MS; 217, 219 (M+, [M+1]+), ¹H NMR (300 MHz, CDCl₃) δ 4.05 (t, 2H, J=6 Hz), 3.1 (t, 2H, J=6 Hz), 2.26 (s, 3H), 2.22 (s, 3H).

### EXAMPLE 5

### Preparation of 2-(4-Bromo-3,5-dimethyl-pyrazol-1-yl)ethylamine Hydrochloride

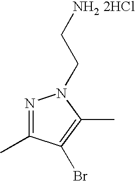

A solution of 2-(4-bromo-3,5-dimethyl-pyrazol-1-yl)ethylamine (0.3 g, 1.3756 mmol) in ether (2 mL) was treated with HCl in ether (13.8 mL, 1 M). The mixture was stirred for 1 h and concentrated. The residue was washed with ether (2×15 mL) and dried under vacuum to give an off-white solid (0.4 g, 100%). Mp 221-222° C. ¹H NMR (300 MHz, CDCl₃) δ 4.52 (t, 2H, J=6.3 Hz), 3.18-3.12 (m, 2H), 2.23 (s, 3H), 2.09 (s, 3H).

### EXAMPLE 6

### Preparation of 5-Methyl-3-phenyl-1H-pyrazole

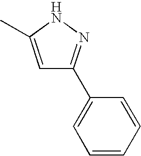

Hydrazine (1.34 mL, 0.0278 mol) was added to a mixture of 1-phenyl-butane-1,3-dione (3 g, 0.0185 mol) and acetic acid (0.5 mL) in ethanol (20 mL). The mixture was refluxed for 4 h and concentrated. The product (2.8 g, 96%) was isolated by column chromatography (50% EtOAc in Hexanes). R_{f}=0.47 (50% EtOAc in Hexane), MS (Electrospray) 159.3 (M+H)⁺, ¹H NMR (300 MHz, CDCl₃) δ 7.76-7.70 (m, 2H), 7.45-7.29 (m, 3H), 6.38 (s, 1H), 2.36 (s, 3H).

### EXAMPLE 7

### Preparation of 2-(5-Methyl-3-phenyl-pyrazol-1-yl)ethylamine

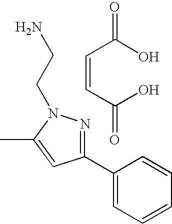

To a suspension of 5-methyl-3-phenyl-1H-pyrazole (1 g, 6.32 mmol) in acetonitrile (4 mL) was added sodium hydroxide (1.01 g, 25.28 mmol). The mixture was stirred under argon for 30 min at room temperature. 2-chloroethylamine hydrochloride (1.1 g, 9.48 mmol.) was added followed by tetrabutylammonium hydrogen sulfate (0.08 g, 0.25 mmol), the reaction mixture was stirred at reflux for 18 h and diluted with ethyl acetate (20 mL). The mixture was filtered and the filtrate concentrated. The residue was purified by column chromatography (5-10% MeOH—CH₂Cl₂). The eluants were concentrated. Reversed phase HPLC was use to separate isomers. A maleic acid salt was obtained by stirring maleic acid (0.055 g) with one of the fractions from HPLC in diethyl (20 mL) ether to give 0.0402 g, 30% of product after concentration under vacuum. mp 131-132° C., ¹H NMR (300 MHz, DMSO) δ 7.83-7.79 (m, 2H), 7.41-7.25 (m, 3H), 6.47 (s, 1H), 6.21 (s, 2H), 4.59 (t, 2H, J=6 Hz), 4.33 (t, 2H, J=6 Hz), 2.36 (s, 3H).

### EXAMPLE 8

### Preparation of 4-(4-bromobenzyl)-3,5-heptanedione

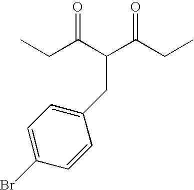

To a solution of 3,5-heptanedione (12.81 g, 0.1 mol) in acetonitrile (200 mL) cooled in an ice-water bath was added 4-bromo-benzylbromide (25 g, 0.1 mol), followed by potassium carbonate (6.91 g, 0.05 mol) slowly. The reaction mixture was heated at 60° C. for 18 hours and concentrated. To the crude residue was added water (100 mL) and the aqueous layer was extracted with ethyl acetate (3×100 mL). The combined organic layers were washed with brine (2×50 mL) and dried over MgSO₄. Filtration and concentration of the organic phase afforded a crude oil residue, which was chromatographed with a solution hexane/ethyl acetate=100/2 to obtain an orange color oil (15.5 g, 52%), which was used in next step without further purification. MS (Electrospray) 297 (M)⁺.

### EXAMPLE 9

### Preparation of 4-(4-bromobenzyl)-3,5-diethyl-1H-pyrazole

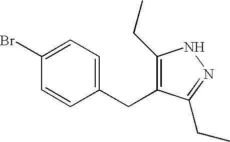

To a solution of Example 8 (15.5 g, 0.052 mol) in ethanol (50 mL) was added hydrazine (2.44 mL, 0.078 mol). The mixture was stirred at room temperature for 18 h and concentrated. To the crude residue was added 1N HCl to pH 2, then the aqueous solution was washed with ethyl acetate (2×25 mL), basified with 1N NaOH to pH 11-12 and extracted with ethyl acetate (3×50 mL). The combined organic layers were washed with water (2×20 mL), brine (20 mL) and dried over MgSO₄. Filtration and concentration of the organic phase afforded a yellowish oil, which was chromatographed with ethyl acetate to obtain a yellowish oil (7 g, 45.8%). MS (Electronspray) 293 (M)⁺, ¹H NMR (300 MHz, CDCl₃) δ 7.37-7.34 (d, 2H), 6.98-6.95 (d, 2H), 3.71 (s, 2H), 2.55-2.47 (q, 4H), 1.183-1.132 (t, 6H).

### EXAMPLE 10

### Preparation of 2-[4-(4-bromobenzyl)-3,5-diethyl-1H-pyrazol-1-yl]ethylamine

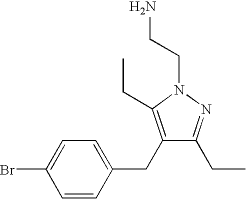

To a suspension of Example 9 (2.93 g, 0.01 mol) in acetonitrile (50 mL) was added sodium hydroxide (1.6 g, 0.04 mol). The mixture was stirred under argon for 30 min at room temperature. 2-chloroethylamine hydrochloride (1.74 g, 0.015 mol) was added, followed by tetrabutylammonium hydrogen sulfate (0.136 g, 0.4 mmol), the reaction mixture was stirred at reflux for 3 h, then diluted with ethyl acetate (50 mL). The mixture was filtered and the filtrate concentrated. The residue was dissolved in ethyl acetate (70 mL), dried over MgSO₄. Filtration and concentration of the organic phase afforded a crude residue which was chromatographed with CH₂Cl₂/CH₃OH/NH₄OH=10:1:0.1 to provide a yellowish oil (1.07 g, 31.8%). R_{f}=0.33 (CH₂Cl₂/CH₃OH=10:1). MS (Electrospray) 339 (M+2)⁺, ¹H NMR (300 MHz, CDCl₃) δ 7.36-7.34 (d, 2H), 6.98-6.95 (d, 2H), 4.06-4.02 (t, 2H), 3.69 (s, 2H), 3.15-3.11 (t, 2H), 2.56-2.42 (q, 4H), 1.14-1.09 (t, 3H), 1.04-0.99 (t, 3H).

### EXAMPLE 11

### Preparation of 3,5-diethyl-4-[(4′-methoxy-1,1′-biphenyl-4-yl)methyl]-1H-pyrazole

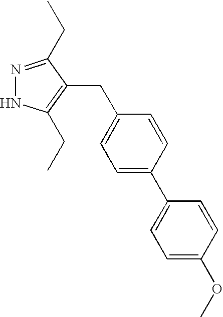

A mixture of Example 9 (879.6 mg, 3 mmol), 4-methoxyphenyl boronic acid (911.8 mg, 6 mmol), PdCl₂(PPh₃)₂ (42.11 mg, 0.06 mmol) and Na₂CO₃ (0.63 mL, 2N) in toluene (15 mL) was heated at 90° C. for 18 h. The reaction mixture was cooled to room temperature, then quenched with water and extracted with ethyl acetate (3×20 mL). The combined organic layers were washed with water (15 mL), brine (15 mL) and dried over MgSO₄ and concentrated. The product was isolated by column chromatography (100% EtOAc) to give a white solid (744 mg, 77%). R_{f}=0.66 (100% EtOAc), MS (Electrospray) 321(M+H)⁺, ¹H NMR (300 MHz, CDCl₃) δ 7.51-7.42 (m, 4H), 7.15-7.13 (d, 2H), 6.97-6.94 (d, 2H), 3.84 (s, 3H), 3.79 (s, 2H), 2.58-2.53 (q, 2H), 1.21-1.16 (t, 6H).

### EXAMPLE 12

### Preparation of 2-{3,5-diethyl-4-[(4′-methoxy-1,1′-biphenyl-4-yl)methyl]-1H-pyrazol-1-yl}ethylamine

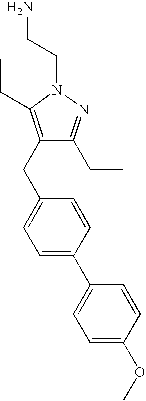

To a suspension of Example 11 (384 mg, 1.2 mmol) in acetonitrile (20 mL) was added sodium hydroxide (192 mg, 4.8 mmol). The mixture was stirred under argon for 30 min at room temperature. 2-chloroethylamine hydrochloride (209 mg, 1.8 mmol) was added, followed by tetrabutylammonium hydrogen sulfate (16.3 mg, 0.048 mmol), the reaction mixture was stirred at reflux for 3 h and acetonitrile was removed. To the residue was added ethyl acetate (40 mL) and water (20 mL). The organic layer was separated and washed with water (10 mL), brine (10 mL) and dried over MgSO₄ Filtration and concentration of the organic phase afforded a crude residue, which was chromatographed with CH₂Cl₂/CH₃OH/NH₄OH=10:1:0.1 to provide white solid (358 mg, 79.9%). R_{f}=0.3 (CH₂Cl₂/CH₃OH=10:1). MS (Electrospray) 364 (M+H)⁺, ¹H NMR (300 MHz, CDCl₃) δ 7.51-7.42 (m, 4H), 7.15-7.12 (d, 2H), 6.96-6.94 (d, 2H), 4.07 (t, 2H), 3.84 (s, 3H), 3.78 (s, 2H), 3.17 (t, 3H), 2.58-2.50 (q, 4H), 1.18-1.13 (t, 3H), 1.07-1.02 (t, 3H).

### EXAMPLE 13

### Preparation of 4-(4-nitrobenzyl)-3,5-heptanedione

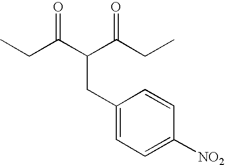

Lithium hydride (25.1 g, 3.15 mole) was suspended in anhydrous DMF (2 L, under argon) and stirred while cooling to −10° C. A solution containing 3,5-heptanedione (500 g, 3.90 mole) in DMF (1 L) was added to the reaction over 1.25 h and stirred for an additional 2 hours while warming to room temperature. The mixture was cooled again (−10° C.) and a solution containing 4-nitrobenzyl bromide (716 g, 3.32 mole) in DMF (2 L) slowly added over 1.25 h. After stirring at room temperature for 18 hours the reaction mixture was diluted with dichloromethane (6 L) and washed with dilute hydrochloric acid (4.5 L, 1.0 N). The aqueous washes were extracted with dichloromethane and the combined organic portions were dried over magnesium sulfate, filtered and concentrated to in vacuo to leave a yellow oil (828 g, 95%). The crude product was used without further purification. ¹H-NMR: (CDCl₃): δ 1.0 (dt, J=1.4, 6.7 Hz, 3H, CH₃), 2.33 (m, 2H, CH₂) 2.51 (2H, CH₂), 3.24 (d, J=7.3, 2H, CH₂), 4.01 (dt, J=1.1, 7.4 Hz, 1H, CH), 7.31 (d, J=7.3 Hz, 2H, Ar), 8.13 (d, J=8.4 Hz, 2H, Ar).

### EXAMPLE 14

### Preparation of Tert-Butyl 2-bromoethylcarbamate

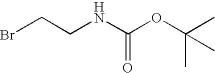

Di-t-butyl dicarbonate (533 g, 2.44 mole) in tetrahydrofuran (THF, 1.5 L) solution and a sodium hydroxide solution, (−1.6 N, 1.5 L) were simultaneously added dropwise to a cold (0° C.), stirred solution of 2-bromoethylamine hydrobromide (500 g, 2.44 mole) in water (500 mL). The bi-phasic mixture was stirred for 18 hours at room temperature, then the reaction was treated with additional 2-bromoethylamine hydrobromide (200 g, 0.98 mole, neat) and sodium hydroxide (60 g, 1.5 mole, solid). After stirring for 4 hours at room temperature the reaction mixture was concentrated in vacuo and the aqueous residue was extracted with ethyl acetate (3×1 L). The combined organic portions were washed with dilute phosphoric acid (10%, 2×500 mL), water (500 mL) and dried over magnesium sulfate. Filtration and concentration in vacuo gave a pale yellow oil (519 g, 95% based on tBOC reagent). The material was used without further purification. ¹H-NMR (DMSO-d₆): δ1.37 (s, 9H, t-Bu), 3.28 (t, J=6.1 Hz, 2H, CH₂,), 3.40 (t, J=5.6 Hz, 2H, CH₂), 7.1 (br d, J=4.9 Hz, 1H, NH,).

### EXAMPLE 15

### Preparation of Tert-Butyl 2-hydrazinoethylcarbamate

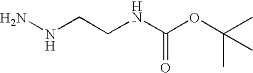

A solution of N-BOC-aminoethyl-2-bromide (450 g, 2.0 mole) in ethanol (1.5 L) was added over a 1.5 hour period to a refluxing solution of hydrazine hydrate (650 g, 12.0 mole) in ethanol (1.5 L). After continuing refluxing for 1 hour the reaction mixture was concentrated in vacuo and the oily residue was dissolved in ether (2 L). The ethereal solution was washed with saturated sodium carbonate/sodium chloride solution (1 L) and the aqueous wash was extracted twice with ether/ethyl acetate (1:1, 1 L). The combined organic portions were dried over sodium sulfate, filtered and concentrated in vacuo to give the product as a clear, pale-yellow oil (235 g, 67%). The material was used without further purification. ¹H-NMR: (DMSO-d₆) δ1.28 (s, 9H, t-Bu), 2.91 (m, 2H, CH₂), 3.25 (br s, 4H, CH₂ NH₂), 6.58 (br s, 1H. NH).

### General Experimental Procedure for Parallel Synthesis Reactions

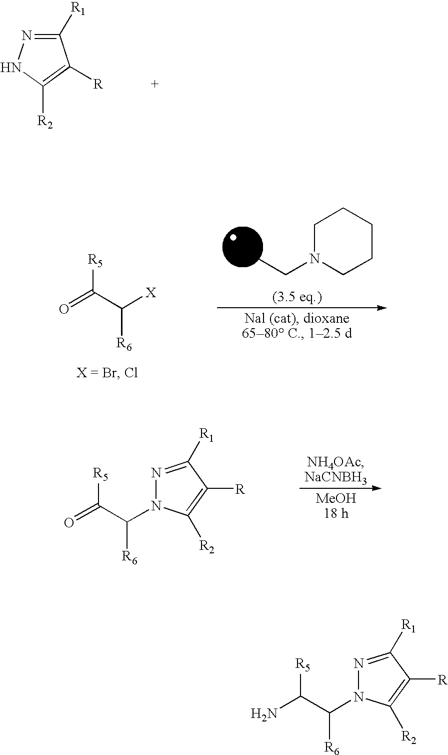

Reactions were carried out in 8-mL glass vials with Teflon-lined screw caps, or in a polypropylene reaction block consisting of a 6×8 matrix of forty-eight 5.6-mL reaction wells, with each reaction well incorporating a 15-45 micron polyethylene frit; reaction blocks of this type are commercially available as FlexChem™ reactor blocks from Robbins Scientific Corporation, Sunnyvale, Calif. The reactor blocks are sealed with rubber gaskets and a clamping device, and can be heated with mixing by rotation in an oven (Robbins Scientific).

### EXAMPLE 16

### Preparation of 3-(1H-pyrazol-1-yl)-2-butanones

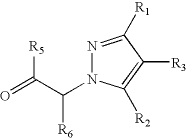

In a typical procedure, solutions of α-bromomethyl and/or α-chloromethyl ketones were prepared as 1.0 M in dioxane, and solutions of pyrazoles (commercially-available or prepared according to methods well known in the art) were prepared as 250 mM in dioxane. To each reaction well in a polypropylene reaction block was added sodium iodide (5 mg), followed by piperidinomethyl polystyrene (200 mg, 0.7 mmol, 3.5 mmol/g, commercially available from NovaBiochem, La Jolla, Calif.), a solution of the desired pyrazole (800 μL, 0.2 mmol), and a solution of the desired α-halomethyl ketone (600 μL, 0.6 mmol). The reaction block was sealed with rubber gaskets and clamped, then heated at 65-80° C. for 1-2.5 days, with mixing by rotation. After allowing the reaction block to cool to room temperature, the block was disassembled, and the reaction well contents were filtered into a collection 96-well deep-well microtiter plate, washing with acetonitrile or dichloromethane. The filtrate solutions were analyzed for purity and correct identity by HPLC/UV/ELSD and LC/MS, and were evaporated to dryness using a multiple sample centrifugal vacuum evaporator.

### EXAMPLE 17

### Preparation of 3-(1H-1-pyrazol-1-yl)-2-butanamines

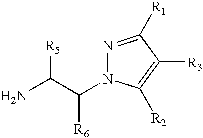

Crude products from the previous step (0.2 mmol scale) were dissolved in 400 μL methanol, and 50 μL of this solution (ca. 15-25 μmol) was added to each reaction well, as desired. Solutions of ammonium acetate in methanol (1.5 M) and sodium cyanoborohydride in methanol (1.0 M) were prepared. To each reaction well was added powdered 4 Å molecular sieves (25 mg), followed by the ammonium acetate solution (167 μL, 250 μmol), and then the reaction mixture was mixed by orbital shaking for 510 min. Sodium cyanoborohydride solution was then added (25 μL, 25 μmol) to each reaction well, and then the reaction block was sealed with a gasket and rotated at room temperature for 18 h. The reaction block was disassembled and the reaction contents reaction well contents were filtered into a collection 96-well deep-well plate, washing with 2×250 μL dichloromethane. Using a well-ventilated fume hood, 6.0 N HCl (20 μL) was added to each filtrate solution, followed by 5.0 NaOH (120 mL) and water (500 mL). After the reaction mixture was mixed for 1 h, the organic phase was removed to a clean vial or a to a well in a 96-well deep-well microtiter plate. The product solutions were analyzed for purity and correct identity by HPLC/UV/ELSD and LC/MS, and were evaporated to dryness using a multiple sample centrifugal vacuum evaporator. For compounds of particular interest, this step was carried out on four-fold scale, and the product was purified by preparative reverse phase HPLC, and characterized by LC/MS and NMR.

### EXAMPLE 18

### Preparation of (2S)-2-[(tert-butoxycarbonyl)amino]propyl Methanesulfonate

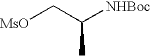

Methanesulfonyl chloride (392 g, 3.42 mole) was slowly added to a cold (−8° C.), stirred solution containing N-BOC−1-alaninol (500 g, 2.85 mole) and triethylamine (361 g, 3.57 mole) in 4 L of dichloromethane. Stirring was continued for 14 hours as the reaction was slowly allowed to warm to room temperature. The reaction mixture was washed with water (2×3 L), dried over magnesium sulfate, filtered and concentrated in vacuo to afford colorless solids (632 g, 88%). The crude product was used in the next step without further purification. ¹H-NMR (DMSO-d₆):δ1.04 (d J=6.7 Hz, 3H, CH₃,), 1.37 (s, 9H, t-Bu), 3.15 (s, 3H, CH₃SO₂), 3.73 (m, 1H, CH), 4.02 (d, J=5.8 Hz, 2H, CH₂,), 6.93 (brd, J=8.0 Hz, 1H, NH).

### EXAMPLE 19

### Preparation of Tert-Butyl (1S)-2-bromo-1-methylethylcarbamate

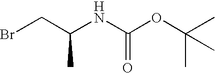

A solution containing N-BOC-I-alaninyl-methanesulfonate (710 g, 2.80 mole) in acetone (6 L) was treated with lithium bromide (730 g) and stirred at room temperature for 18 hours. The reaction mixture was concentrated to dryness in vacuo and the residue partitioned between water (2 L) and dichloromethane (2 L). The layers were separated and the organic layer was dried over magnesium sulfate, filtered and concentrated in vacuo to afford off-white solids (387 g, 58%). The crude product was used in the next step without further purification. ¹H-NMR (DMSO-d₆): δ 1.09 (d, J=6.6 Hz, 3H, CH₃,), 1.37 (s, 9H, t-Bu), 3.41 (d, J=6.0 Hz, 2H, CH₂), 3.66 (m, 1H, CH), 6.95 (Br s, J=7.9 Hz, 1H, NH,).

### EXAMPLE 20

### Preparation of Tert-Butyl (1S)-2-hydrazino-1-methylethylcarbamate

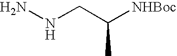

A solution containing N-BOC-1-alaninyl bromide (200 g, 0.84 mole) in ethanol (425 mL) was added over a 1 hour period to a refluxing solution of hydrazine hydrate (252 g, 5.04 mole) in ethanol (425 mL). Refluxing was continued for 1 hour and the reaction mixture concentrated in vacuo. The residual oil was dissolved in ether (1 L) and the solution washed with a saturated sodium carbonate/sodium chloride solution (1×700 mL, 1×300 mL). The aqueous wash was extracted twice with ether (1×1 L, 1×500 mL) and the combined portions were dried over sodium sulfate. Filtration and concentration in vacuo gave the hydrazide as a pale yellow oil (114 g, 72%) which was used for the next step without further purification. ¹H-NMR (DMSO-d₆): δ 0.97 (d, J=6.5 Hz, 3H, CH₃,), δ 1.36 (s, 9H, t-Bu), 2.47 (d, J=6.2 Hz, 2H, CH₂,) 3.45 (br s, 2H, NH₂), 3.60 (m, 1H, CH), 6.58 (br d, J=8.5 Hz, 1H, NH).

### EXAMPLE 21

### Preparation of Benzyl 4-[(1-{(2R)-2-[(tert-butoxycarbonyl)amino]propyl}-3,5-dimethyl-1H-pyrazol-4-yl)methyl]-1-piperidinecarboxylate

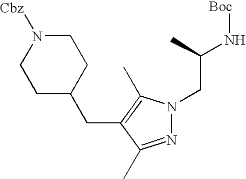

### Step 1. Preparation of Benzyl 4-(hydroxymethyl)-1-piperidinecarboxylate

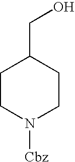

To a solution of 50 g of ethyl 4-piperidinecarboxylate (318 mmol) in 600 mL ether was added aqueous K₂CO₃ (630 mL, 2M) at 0° C. and then benzyl chloridocarbonate (70.5 g, 413.5 mmol) was added drop wise into the stirred mixture. Following completion of the addition, the mixture was stirred an additional 20 min at 0° C. The mixture was then extracted with ether, the ether dried over MgSO₄ and concentrated in vacuo to give 115.0 g of crude product.

This material was dissolved in 200 mL THF and LiB(Et)₃H (450 mL, 1M) was added slowing via a cannula at 0° C. The solution was stirred at 0° C. for 1.5 h and quenched with NH₄Cl. The mixture was concentrated to ⅓ volume, extracted with EtOAc and the organic extract was dried over MgSO₄ and concentrated in vacuo. The product was purified by chromatography on a Biotage unit eluting with 1:1; Hexanes: EtOAc. The overall yield was 82.28 g (87%).

### Step 2. Preparation of Benzyl 4-(bromomethyl)-1-piperidinecarboxylate

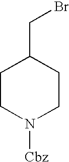

The product of step 1 (50 g, 0.20 mol) was dissolved in 1 L of methylene chloride and 88.9 g (0.211 mol) of dibromo(triphenyl)phosphorane was added at rt and stirred for 1 h. The mixture was concentrated in vacuo, and the product isolated after purification by column chromatography eluting with a gradient solvent mixture of hexanes/CH₂Cl₂. Yield: 41.52 g (82.3%).

### Step 3. Preparation of Benzyl 4-(2-acetyl-3-oxobutyl)-1-piperidinecarboxylate

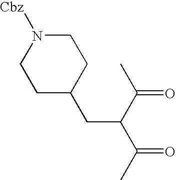

The product of step 2 (26.89 g, 86.1 mmol) was dissolved in DMF (400 mL) and sodium hydride (4.13 g 172.3 mmol, [6.92 g of 60%]) was added. To this stirred mixture at rt was slowly added 17.25 g (172.2 mmol)pentane-2,4-dione. The mixture was stirred for 15 h and then carefully quenched with water and extracted with ether. The ether solution was dried over MgSO₄, concentrated in vacuo, and purified using a Biotage SGC column, eluting with 2:1 hex/EtOAc, to give 10.2 g (36%).

### Step 4. Preparation of Benzyl 4-[(1-{(2R)-2-[(tert-butoxycarbonyl)amino]propyl}-3,5-dimethyl-1H-pyrazol-4-yl)methyl]-1-piperidinecarboxylate

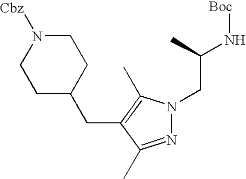

To a solution of the product of step 3 (3.5 g, 10.56 mmol) in absolute ethanol was added Example 20 (4 g, 21.13 mmol). The mixture was stirred under argon for 16 hours at reflux. The reaction mixture was concentrated in vacuo and purified via flash chromatography using 50% EtOAc/Hexanes to yield a white solid (2.68 g, 5.52 mmol, 53%). ¹H NMR (CDCl₃, 300 MHz): δ 7.27 (s, 5H), 5.23 (s, 2H), 5.18 (m, 1H), 4.25 (m, 2H), 3.46 (m, 4H), 3.09 (m, 4H), 2.43 (m, 2H), 2.09 (s, 3H), 1.95 (s, 3H), 1.45 (m, 3H), 1.40 (s, 9H). MS (electronspray) M+H⁺ 485. R_{f}=0.3 (50% EtOAc/Hexanes).

### EXAMPLE 22

### Preparation of Tert-Butyl (1R)-2-[3,5-dimethyl-4-(4-piperidinylmethyl)-1H-pyrazol-1-yl]-1-methylethylcarbamate

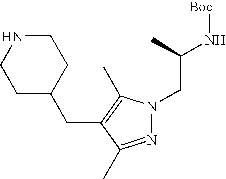

To a dry round bottom flask was added 10% palladium on carbon (0.268 g) under an argon atmosphere. Example 21 (2.68 g, 5.53 mmol) in EtOAc (20 mL) was added to this under a blanket of argon. The argon was evacuated and the reaction mixture was placed under a hydrogen atmosphere. After 18 hours, the mixture was removed from the hydrogen atmosphere and filtered through a pad of Celite. The Celite was washed with EtOAc and the filtrate was concentrated to yield a yellowish oil (1.63 g, 4.65 mmol, 85%). ¹H NMR (CDCl₃, 300 MHz): δ 5.20 (m, 1H), 4.20 (m, 2H), 3.40 (m, 4H), 2.98 (m, 4H), 2.43 (m, 2H), 2.09 (s, 3H), 1.95 (s, 3H), 1.45 (m, 3H), 1.40 (s, 9H). MS (electronspray) M+H⁺ 351. R_{f}=0.1 (10% MeOH/dichloromethane).

### EXAMPLE 23

### Preparation of Tert-Butyl (1R)-2-(4-{[1-(2,2-dimethylpropanoyl)-4-piperidinyl]methyl}-3,5-dimethyl-1H-pyrazol-1-yl)-1-methylethylcarbamate

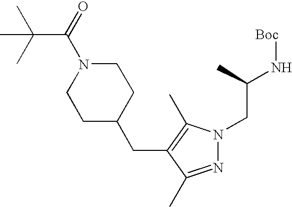

To solution of Example 22 (0.3 g, 0.86 mmol) in dichloromethane (2 mL) was added poly-4-vinyl-pyridine (0.74 g, 2.58 mmol) followed by trimethylacetyl chloride (0.15 mL, 1.71 mmol), the reaction mixture stirred 18 hours at room temperature. The reaction mixture was filtered through a coarse filter frit and the filtrate was concentrated to yield the product (0.336 g, 0.774 mmol, 90%). ¹H NMR (CDCl₃, 300 MHz): δ 5.20 (m, 1H), 4.32 (m, 2H), 3.43 (m, 4H), 3.20 (m, 4H), 2.43 (m, 2H), 2.09 (s, 3H), 1.95 (s, 3H), 1.45 (m, 3H), 1.40 (s, 9H) 1.50 (s, 9H). MS (electronspray) M+H⁺ 435.

### EXAMPLE 24

### Preparation of (2R)-1-(4-{[1-(2,2-dimethylpropanoyl)-4-Piperidinyl]methyl}-3,5-dimethyl-1H-pyrazol-1-yl)-2-propanamine Trifluoroacetic Acid Salt

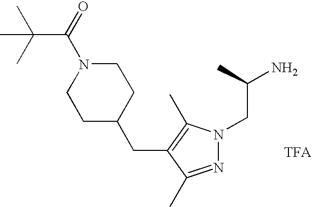

A solution of Example 23 (0.336 g, 0.774 mmol) in dichloromethane (5 mL) was treated dropwise with concentrated trifluoroacetic acid (5 mL). The mixture was stirred for 1 h and concentrated. The residue was washed twice with ether (7 mL) and dried under vacuum to give a yellowish solid (0.258 g, 0.774, 100%). MS (electronspray) M+H⁺ 335.

### EXAMPLE 25

### Preparation of Tert-Butyl (1S)-2-(3,5-diethyl-4-[4-(methylamino)benzyl]-1H-pyrazol-1 yl}-1-methylethylcarbamate

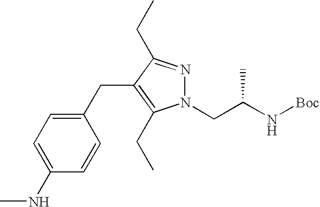 tert-Butyl (1S)-2-[4-(4-aminobenzyl)-3,5-diethyl-1H-pyrazol-1-yl]-1-methylethyl-carbamate was prepared in two steps from Example 13 and Example 15 using the method of Example 21, step 4, followed by reduction in ethanol using 10% Pd on C in a Parr apparatus under 60 psi of hydrogen.

To a solution of the compound thus obtained (95 mg, 065 mmol) in 2 mL of methanol was added paraformaldehyde (27 mg, 0.91 mmol) and 0.2 mL of sodium methoxide (25 wt % in methanol). The reaction solution was stirred at room temperature under argon for 16 hours. At this time sodium borohydride (29 mg, 0.78 mmol) was added and the resulting mixture was heated to reflux for three hours then cooled to room temperature and quenched with 1N aqueous sodium hydroxide. The aqueous solution was extracted with ethyl acetate. Then the organic phase was washed with brine, dried over anhydrous sodium sulfate, filtered, and concentrated in-vacuo to provide a clear, colorless oil (260 mg, quant.): ES-MS m/z 401 ((M+H)⁺); ¹H NMR (d₆-DMSO) δ 0.91-0.96 (m, 6H), δ 1.0 (t, 3H), 1.31 (s, 9H), 2.31 (q, 2H), 2.51-2.57 (m, 2H), 2.59 (s, 3H), 3.50 (s, 2H), 3.73-3.97 (m, 3H), 5.35 (br. s, 1H), 6.37-6.40 (d, 2H), 6.76-6.78 (d, 2H).

### EXAMPLE 26

### Preparation of Tert-Butyl (1S)-2-(3,5-diethyl-4-{4-[methyl(methylsulfonyl)amino]benzyl}-1H-pyrazol-1-yl)-1-methylethylcarbamate

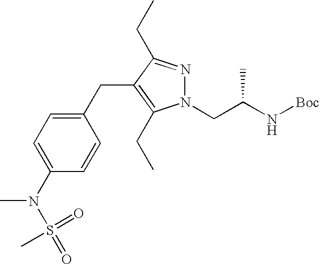

To a solution of Example 25 (260 mg, 0.65 mmol) in 3 mL of dichloromethane was added methanesulfonyl chloride (0.05 mL, 0.71 mmol) and pyridine (0.11 mL, 1.3 mmol). The reaction solution was stirred at room temperature under argon for 16 hours then concentrated in-vacuo. The resulting residue was purified through flash column chromatography with 1:1 hexanes:ethyl acetate as the eluant to provide a clear, colorless oil (232 mg, 75%): ES-MS m/z 479 ((M+H)⁺); ¹H NMR (d₆-DMSO) δ 0.92-0.97 (m, 6H), δ 0.99-1.04 (t, 3H), 1.34 (s, 9H), 2.30-2.38 (q, 2H), 2.52-2.61 (m, 2H), 2.88 (s, 3H), 3.18 (s, 3H), 3.69 (s, 2H), 3.79-3.96 (m, 3H), 6.79-6.82 (d, 1H), 7.08-7.10 (d, 2H), 7.25-7.28 (d, 2H).

### EXAMPLE 27

### Preparation of N-[4-({1-[(2S)-2-aminopropyl]-3,5-diethyl-1H-Pyrazol-4-yl}methyl)phenyl]-N-methylmethanesulfonamide dihydrochloride

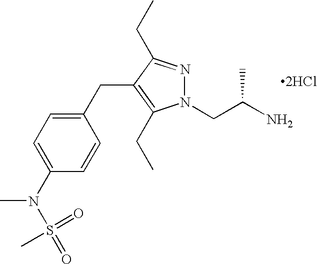

To a solution of Example 26 (230 mg, 0.48 mmol) in 5 mL of dichloromethane was added 5 mL of hydrochloric acid (2N in diethyl ether). The reaction solution was stirred at room temperature for 20 hours then concentrated in-vacuo to provide a pale yellow solid (185 mg, 85%): ES-MS m/z 379 ((M+H)⁺); ¹H NMR (d₆-DMSO) δ 0.95 (t, 3H), 1.05 (t, 3H), 1.13-1.15 (d, 3H), 2.32-2.41 (q, 2H), 2.55-2.63 (m, 2H), 2.89 (s, 3H), 3.18 (s, 3H), 3.57-3.66 (m, 1H), 3.72 (s, 2H), 4.05-4.20 (m, 2H), 7.10-7.13 (d, 2H), 7.27-7.30 (d, 2H), 8.14 (br. s, 2H).

### EXAMPLE 28

### Preparation of 1-methylcyclopropanecarbonyl chloride

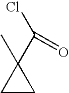

To a solution of 1-methylcyclopropane-1-carboxylic acid (82 mg, 0.82 mmol) in dichloromethane (2.0 mL) was added oxalyl chloride (0.07 mL, 0.82 mmol) and few drops of N,N-dimethylformamide. The resulting mixture was then stirred at room temperature for 1 hour. The resulting acid chloride was used without further purification in the next step.

### EXAMPLE 29

### Preparation of Tert-Butyl (1R)-2-[4-(4-amino-3-chlorobenzyl)-3,5-diethyl-1H-pyrazol-1-yl]-1-methylethylcarbamate

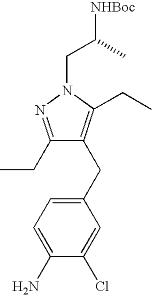

To a solution of Tert-Butyl (1R)-2-[4-(4-aminobenzyl)-3,5-diethyl-1H-pyrazol-1-yl]-1-methylethylcarbamate (as described in Example 25, 2.2 gm, 5.7 mmol) in 10 mL of carbon tetrachloride was added acetic acid (5.0 mL) and stirred for 15 minutes. N-Chlorosuccinimide (801 mg, 5.98 mmol) was then added and the resulting solution was stirred at room temperature for 18 hours. Aqueous ammonium hydroxide and water were added and the mixture was extracted twice with dichloromethane. The combined organic layers were dried over anhydrous sodium sulfate and concentrated under reduced pressure. The resulting residue was purified by flash chromatography to give the product (780 mg, 32.5%): MS (electron spray) 421 (M+H)⁺; ¹H NMR (300 MHz, CDCl₃) δ 6.95 (s, 1H), 6.75 (d, 1H), 6.63 (d, 1H), 5.48 (br s, 1H), 3.90-4.10 (m's, 3H), 3.61 (m, 2H), 2.40-2.50 (m's, 4H), 1.41 (s, 9H), 1.0-1.15 (m's, 6H).

### EXAMPLE 30

### Preparation of Tert-Butyl (1R)-2-(4-{3-chloro-4-[(2,2-dimethylpropanoyl)amino]benzyl}-3,5-diethyl-1H-pyrazol-1-yl)-1-methylethylcarbamate

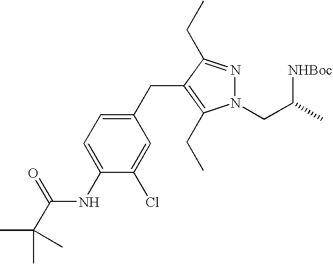

To a stirred solution of Example 29 (164 mg, 0.39 mmol) in dichloromethane (2 mL) was added poly-4-vinyl pyridine (86 mg, 0.78 mmol) and trimethylacetyl chloride (0.056 mL, 0.47 mmol). The resulting solution was stirred at room temperature for 3 hours. The mixture was then filtered and washed with water, dried over anhydrous sodium sulfate and concentrated under reduced pressure. The resulting residue was purified with flash chromatography (Biotage flash 40M) using 1:1 hexane:ethyl acetate to afford product (95 mg, 49%): MS (electron spray) 505.2 (M+H)⁺; ¹H NMR (300 MHz, CDCl₃) δ 8.26 (d, 1H), 7.91 (br s, 1H), 7.04 (s, 1H), 6.98 (d, 1H), 5.45 (br s, 1H), 3.96-4.13 (m, 1H), 3.68 (s, 2H), 2.39-2.56 (m, 4H), 1.42 (s, 9H), 1.38 (s, 9H), 1.28 (d, 3H), 1.02-1.15 (m's, 6H).

### EXAMPLE 31

### Preparation of N-[4-({1-[(2R)-2-aminopropyl]-3,5-diethyl-1H-pyrazol-4-yl}methyl)-2-chlorophenyl]-2,2-dimethylpropanamide Dihydrochloride

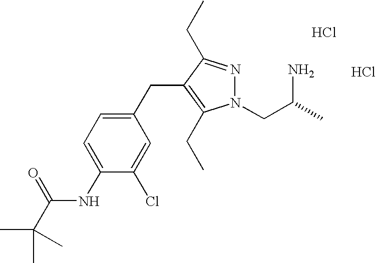

To a solution of Example 30 (95 mg, 0.19 mmol) in dichloromethane (1.5 mL) was added hydrochloric acid (2.0M in ether, 1.88 mL). The resulting solution was stirred at room temperature for 18 hours and then concentrated in vacuo to afford product (69 mg, 76%): MS(electron spray) 505 (M+H)⁺; ¹H NMR (300 MHz, CDCl₃) δ 8.23 (d, 1H), 7.91 (s, 1H), 7.04 (s, 1H), 6.89 (d, 1H), 5.45 (brs, 1H), 4.10 (br, 3H), 3.68 (s, 2H)(m, 4H)(s, 1H)), 1.42 (s, 9H), 1.35 (s, 9H), 1.27 (d, 3H), 1.12 (m, 6H).

### EXAMPLE 31

### Preparation of N-[4-({1-[(2R)-2-aminopropyl]-3,5-diethyl-1H-pyrazol-4-yl}methyl)-2-chlorophenyl]-2,2-dimethylpropanamide dihydrochloride

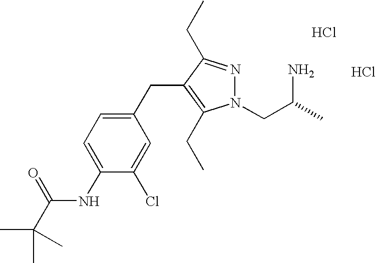

To a solution of Example 30 (95 mg, 0.19 mmol) in dichloromethane (1.5 mL) was added hydrochloric acid (2.0M in ether, 1.88 mL). The resulting solution was stirred at room temperature for 18 hours and then concentrated under reduced pressure to afford product (69 mg, 76%). MS (electron spray) 404.2 (M+H)⁺; ¹H NMR (300 MHz, CDCl₃) δ 8.93 (s, 1H), 7.30 (d, 1H), 7.17 (s, 1H), 7.01 (d, 1H), 4.10-4.15 (m, 2H), 3.72 (s, 2H), 3.58-3.62 (m, 1H), 2.55-2.62 (q, 2H), 2.35-2.42 (q, 2H), 1.20 (s, 9H), 1.12 (d, 3H), 1.02-1.07 (d, 3H), 0.93-0.98 (d, 3H).

### EXAMPLE 32

### Preparation of Ethyl 2-(methoxyimino)-4-oxopentanoate

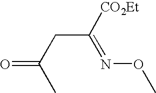

A solution/suspension of Ethyl 2,4-oxovalerate (11.3 gm, 71.2 mmol) and methoxylamine-hydrochloride (5.83 gm, 69.8 mmol), 3 Å molecular sieves (70 g) in ethanol (70 mL) was stirred overnight. The mixture was filtered and concentrated and the crude product was dissolved in diethyl ether and washed with saturated aqueous sodium bicarbonate. The organic layer was dried (sodium sulfate) and concentrated. The residue was purified (Biotage Flash 40, 5 to 50% ethyl acetate/hexanes) to afford the product (4.76 g, 36%) as a clear oil: MS (electron spray) 188.0 (M+H)⁺; ¹H NMR (300 MHz, CDCl₃) δ 4.32 (q, 2H), 4.06 (s, 3H), 3.71 (s, 2H), 2.20 (s, 3H), 1.35 (t, 3H).

### EXAMPLE 33

### Preparation of Ethyl 3-(4-fluorobenzyl)-2-(methoxyimino)-4-oxopentanoate

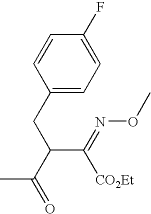

Potassium carbonate (4.22 gm, 30.5 mmol) was added to a solution of Example 32 (4.75 gm, 25.4 mmol) in dimethylformamide (60 mL). 4-Fluoro benzyl bromide (3.16 mL, 25.4 mmol) was added and the mixture was stirred overnight at room temperature. The resulting bright red mixture was diluted with water and ethyl acetate. The layers were separated and the organic layer was washed with 1N hydrochloric acid. The aqueous layers were extracted with ethyl acetate and the combined extracts were dried (sodium sulfate) and concentrated to afford red oil. Purification of the residue by flash chromatography (silica gel) gave product (1.82 g, 47%) as a clear oil. MS (electron spray) 296.0 (M+H)⁺; ¹H NMR (300 MHz, CDCl₃) δ 7.10-7.00 (m, 2H), 6.95-6.85 (m, 2H), 4.25-4.15 (m's, 3H), 4.00 (s, 3H), 3.32 (dd, 1H), 2.92 (dd, 1H), 2.04 (s, 3H), 1.23 (t, 3H).

### EXAMPLE 34

### Preparation of Ethyl 4-(4-fluorobenzyl)-3-methyl-1H-pyrazole-5-carboxylate

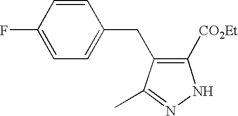

Hydrazine hydrochloride (784 mg, 11.5 mmol) was added to a solution/suspension of Example 33 (2.6 g, 8.8 mmol) in ethanol (40 mL) and 3 Å molecular sieves (2.5 g). The mixture was heated at 80° C. for 3 h. The mixture was filtered through celite and the filtrate was concentrated. The crude material was dissolved in ethyl acetate and washed with saturated aqueous sodium bicarbonate. The aqueous layer was extracted with ethyl acetate and the combined ethyl acetate extracts were dried (sodium sulfate) and concentrated. Purification of the residue by flash chromatography afforded product (2.5 mg, 59%) as a white solid: MS (electrospray) 263.1 (M+H)⁺; ¹H NMR (300 MHz, CDCl₃) δ 7.15-7.05 (m, 2H), 7.00-6.85 (m, 2H), 4.35 (q, 2H), 4.07 (s, 2H), 3.90 (br s, 1H), 2.22 (s, 3H), 1.33 (t, 3H).

### EXAMPLE 35

### Preparation of N,N-dibenzyl-N-(2-iodoethyl)amine Hydroiodide

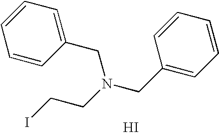

A mixture of N-(2-chloroethyl)dibenzylamine-hydrochloride (12.0 gm, 40.5 mmol), dry acetone (10 mL) and sodium iodide (15.2 gm, 101 mmol) was refluxed overnight. The mixture was cooled and the product was collected by filtration. Diethyl ether was added to the filtrate, causing precipitation of product which was again collected by filtration. The combined solids were extracted with hot ethanol (3×). The product precipitated upon cooling of the ethanol extracts. Filtration and drying provided the iodide (3 gm, 18%) as a white solid: MS (electron spray) 352.1 (M+H)⁺.

### EXAMPLE 36

### Preparation of Ethyl 1-[2-(dibenzylamino)ethyl]-4-(4-fluorobenzyl)-3-methyl-1H-pyrazole-5-carboxylate (36a) and Ethyl 1-[2-(dibenzylamino)ethyl]-4-(4-fluorobenzyl)-5-methyl-1H-pyrazole-3-carboxylate (36b)

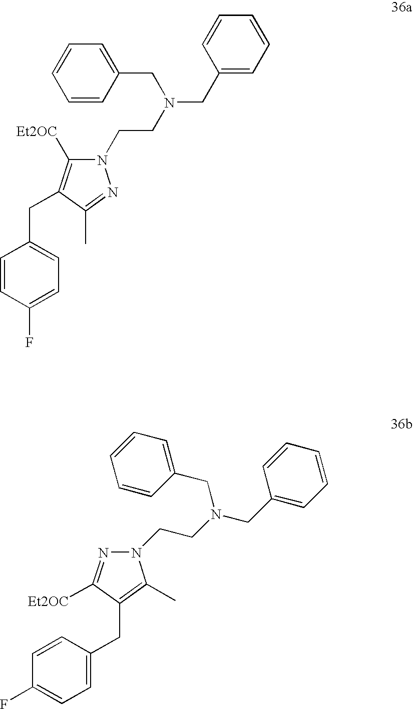

Sodium hydride (41 mg, 1.03 mmol) in toluene (3.6 mL) was added dropwise to a suspension of Example 34 (130 mg, 0.49 mmol) in toluene (3.6 mL) at room temperature. The resulting mixture was heated to 60° C. for 1 h. Example 35 (230 mg, 0.49 mmol) was added and the mixture was heated at 120° C. for 6 h. The reaction mixture was cooled and diluted with ethyl acetate and water. The layers were separated and the organic layer was dried (sodium sulfate). Concentration under reduced pressure gave crude material which was purified by flash chromatography (silica gel, 7:3 hexane/ethyl acetate) to afford Example 36a (35 mg, 15%); MS (electron spray) 486.3 (M+H)⁺. Example 36b: (85 mg, 36%); MS (electron spray) 486.3 (M+H)⁺.

### EXAMPLE 37

### Preparation of Ethyl 1-(2-aminoethyl)-4-(4-fluorobenzyl)-5-methyl-1H-pyrazole-3-carboxylate, 2-butenedioic Acid Salt (1:1)

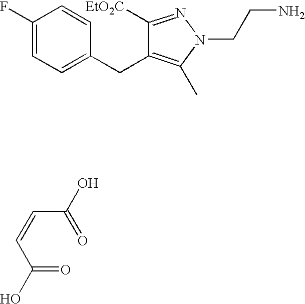

A 250 mL Parr shaker flask containing palladium on carbon (8 mg, 10% wt) was degassed with Argon. A solution of Example 36a (80 mg, 0.16 mmol) in ethanol (3.2 mL) was added, followed by acetic acid (20 μL). Hydrogen was introduced and the mixture was vigorously shook for 48 h (a second portion of acetic acid (10 μL) was added after 24 h). The reaction mixture was filtered, concentrated, and dissolved in ethyl acetate. The organic layer was washed with saturated aqueous sodium bicarbonate and dried (sodium sulfate). Concentration and purification of the crude material by flash chromotography (silica gel, 10/90 methanol/dichloromethane) provided the amine (20 mg, 41%).

Maleic acid (7.6 mg, 0.65 mmol) was added to a solution of the amine (20 mg, 0.65 mmol) in dry diethyl ether (2.5 mL). The mixture was stirred for 2 h and filtered, giving a white solid which was washed with diethyl ether (3×). Drying gave product (16 mg, 60%) as a white solid: MS (electron spray) 306.2 (M+H)⁺; ¹H NMR (300 MHz, DMSO-d₆) δ 7.87 (br s, 3H), 7.20-7.00 (m, 4H), 6.00 (s, 2H), 4.33 (t, 2H), 4.21 (q, 2H), 4.00 (s, 2H), 3.25 (t, 2H), 2.24 (s, 3H), 1.21 (t, 3H).

### EXAMPLE 38

### Preparation of Ethyl 1-(2-aminoethyl)-4-(4-fluorobenzyl)-3-methyl-1H-pyrazole-5-carboxylate, 2-butenedioic Acid Salt (1:1)

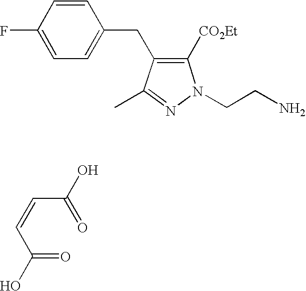

A Parr shaker flask containing palladium hydroxide (30 mg, 20% wt), a solution of Example 36b (60 mg, 0.124 mmol) in ethanol (0.8 mL) and hydrogen was vigorously shook for 18 h. The reaction mixture was filtered through celite, concentrated and purified by flash chromatography (silica gel, 1/99 to 10/90 methanol/dichloromethane) to afford the provided the amine (12 mg, 32%).

Maleic acid (7.6 mg, 0.65 mmol) was added to a solution of the amine (20 mg, 0.65 mmol) in dry diethyl ether (2.5 mL). The mixture was stirred for 2 h and filtered, giving a white solid which was washed with diethyl ether (3×). Drying gave product (16 mg, 60%) as a white solid: MS (electron spray) 306.2 (M+H)⁺.

### EXAMPLE 39

### Preparation of Ethyl 1-{2-[(tert-butoxycarbonyl)amino]ethyl-5-ethyl}-4-(4-fluorobenzyl)-1H-pyrazole-3-carboxylate (39a) and Ethyl 1-{2-[(tert-butoxycarbonyl)amino]ethyl}-3-ethyl-4-(4-fluorobenzyl)-1H-pyrazole-5-carboxylate (39b)

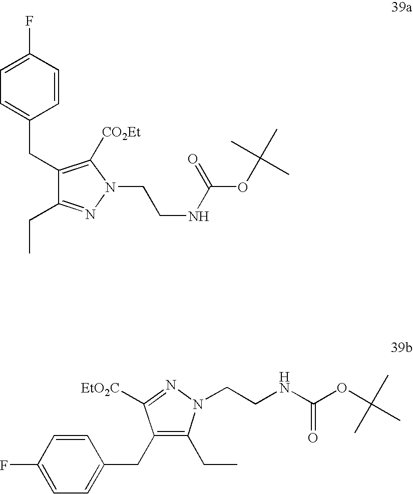

A mixture of ethyl 5-ethyl-4-(4-fluorobenzyl)-1H-pyrazole-3-carboxylate, prepared by the method described for Example 34 (1.28 gm, 4.63 mmol), cesium carbonate (4.53 gm, 13.9 mmol) in dry dimethylformamide (30 mL) was stirred at room temperature for 10 min. tert-Butyl 2-bromoethylcarbamate (Example 4, 91.6 gm, 6.96 mmol) was added and the mixture was stirred overnight. Water (20 mL) was added, followed by 1N hydrochloric acid (10 mL). The aqueous layer was extracted with diethyl ether (3×). The combined organic layers were dried (sodium sulfate) and concentrated under reduced pressure. The crude material was purified by flash chromatography (Biotage Flash 40 system) to afford 39a (1.33 gm, 68%) and 39b (350 mg, 18%).

39a (DMSO, 500 MHz) δ: 0.96 (t, 3H), 1.10 (t, 3H), 1.24 (s, 9H), 2.35 (q, 2H), 3.19 (q, 2H), 3.91 (s, 2H), 4.14 (q, 2H), 4.34 (t, 2H), 6.78 (t, 1H), 6.93-7.07 (m's, 4H).

39b (DMSO, 500 MHz) δ: 0.85 (t, 3H), 1.11 (t, 3H), 1.26 (s, 9H), 2.51 (q, 2H), 3.20 (q, 2H), 3.92 (s, 2H), 4.03 (t, 2H), 4.07 (q, 2H), 6.92-7.07 (m's, 4H).

### EXAMPLE 40

### Preparation of Ethyl 1-(2-aminoethyl)-3-ethyl-4-(4-fluorobenzyl)-1H-pyrazole-5-carboxylate trifluoroacetic Acid Salt (40a) and Ethyl 1-(2-aminoethyl)-5-ethyl-4-(4-fluorobenzyl)-1H-pyrazole-3-carboxylate trifluoroacetic Acid Salt (40b)

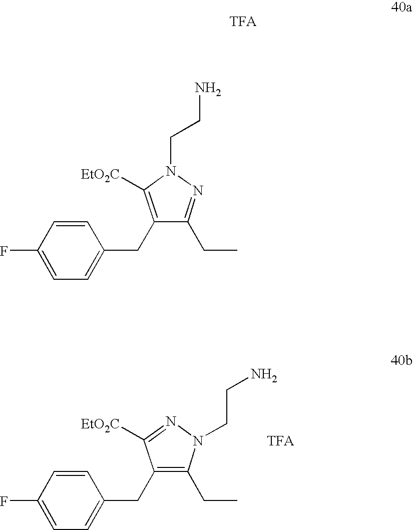

Trifluoroacetic acid (1.5 mL) was added to a solution of Example 35a (100 mg, 0.239 mmol) in dichloromethane (3 mL). The mixture was stirred at room temperature for 2 h and then concentrated under reduced pressure to afford product 40a (103 mg, 99%). MS (electron spray) 320.1 (M+H)⁺; (DMSO, 300 MHz) δ 1.05 (t, 3H), 1.17 (t, 3H), 2.4-2.5 (m, 2H), 3.2-3.25 (m, 2H), 4.00 (s, 2H), 4.23 (q, 2H), 4.62 (t, 2H), 7.0-7.2 (m's, 4H), 7.90 (br s, 3H).

The isomeric Example 40b was prepared according to the same procedure. Yield: 92 mg, 89%; MS (electron spray) 320.1 (M+H)⁺.

### EXAMPLE 41

### Preparation of 1-acetyl-2-oxopropyl benzoate

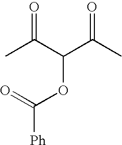

Benzoic acid (3.00 g, 24.6 mmol) was dissolved in DMF (100 mL). Powdered KOH (1.43 g, 24.6 mmol) was added and the resulting mixture heated at 50° C. for 1 h. To the resulting-solution was added neat 3-chloro-2,4-pentanedione (2.93 mL, 24.6 mmol). The reaction was stirred for 18 h at 50° C., cooled to rt and quenched with water (400 mL). The resulting mixture was extracted with Et2O, and the resulting organic layers were combined, washed with saturated aqueous ammonium chloride, dried (MgSO4), and concentrated to a light colored oil (4.81 g, 89%). R_{f}=0.27 (10:90 EtOAc:hex (v/v)); ESLC-MS m/z=221 (MH⁺); ¹H NMR (DMSO-d₆) δ 2.33 (s, 6H), 5.92 (s, 1H), 7.55-7.62 (m, 2H), 7.69-7.76 (m, 1H), 8.05-8.10 (m, 2H) ppm; {¹H}¹³C NMR (DMSO-d₆) δ27.68, 84.73, 128.94, 129.55, 134.06, 199.58.

### EXAMPLE 42

### Preparation of 1-[2-[(tert-butoxycarbonyl)amino]ethyl]-3,5-dimethyl-1H-pyrazol-4-yl benzoate

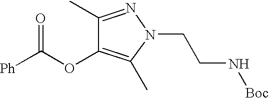

To a solution of Example 41 (4.81 g, 21.8 mmol) in EtOH (50 mL) was added a solution of N-Boc-2-hydrazidoethylamine (6.88 g, 40 mmol) in EtOH (50 mL). The resulting solution was heated to reflux for 1 h, cooled to rt and concentrated to an oily residue. The residue was partitioned between water and EtOAc. The organic layer was collected and the aqueous layer extracted with EtOAc. The combined organic layers were dried (MgSO4) and concentrated. The oily residue was purified on silica using 50:50 EtOAc:hexane (v/v) as eluant to yield, after concentration, an orange solid (6.70 g, 86%). R_{f}=0.71 (EtOAc); ESLC-MS m/z=360 (MH⁺); ¹H NMR (DMSO-d₆) δ1.37 (s, 9H), 1.99 (s, 3H), 2.08 (s, 3H), 3.17-3.25 (m, 2H), 3.94-4.01 (m, 2H), 6.91-6.97 (m, 1H), 7.55-7.63 (m, 2H), 7.70-7.77 (m, 1H), 8.08-8.13 (m, 2H).

### EXAMPLE 43

### Preparation of Tert-Butyl 2-(4-hydroxy-3,5-dimethyl-1H-pyrazol-1-yl)ethylcarbamate

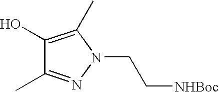

Example 42 (6.70 g, 18.7 mmol) was dissolved in a mixture of EtOH (50 mL). To this solution was added a 1.0 M solution of NaOH in H₂O (50 mL, 50 mmol). The resulting solution was stirred for 3 h at rt and then poured into a saturated solution of sodium chloride in water. The resulting mixture was extracted with EtOAc and the combined organic extracts were dried (MgSO4) and concentrated to a solid. The solid was stirred in Et2O and the suspended solid collected by filtration and dried to yield a white solid (2.14 g, 45%). R_{f}=0.38 (EtOAc); ¹H NMR (DMSO-d₆) δ1.36 (s, 9H), 1.97 (s, 3H), 2.03 (s, 3H), 3.07-3.15 (m, 2H), 3.77-3.85 (m, 2H), 6.81-6.88 (m, 1H), 7.44 (s, 1H).

### EXAMPLE 44

### Preparation of Tert-Butyl 2-[4-(2-ethylbutoxy)-3,5-dimethyl-1H-Pyrazol-1-yl]ethylcarbamate

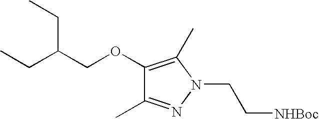

To a solution of Example 43 (60 mg, 0.29 mmol) in DMF (1 mL) was added Cs₂CO₂ (190 mg, 0.60 mmol) followed by 1-bromo-2-ethylbutane (80 μL, 0.58 mmol). The resulting mixture was heated with stirring to 50° C. for 18 h then cooled to rt. The reaction was diluted with water and extracted with EtOAc. The resulting organic layers were combined, dried (MgSO₄), concentrated, and purified on silica using 2:1. EtOAc:hexane (v/v) to yield, after concentration, a white solid (72 mg, 73%). R_{f}=0.62 (EtOAc); ESLC-MS m/z=340 (MH⁺); ¹H NMR (DMSO-d₆) δ 0.88 (t, J=7.2 Hz, 6H), 1.27-1.50 (m, 14H), 2.02 (s, 3H), 2.08 (s, 3H), 3.10-3.18 (m, 2H), 3.60 (d, J=4.8 Hz, 2H), 3.82-3.89 (m, 2H), 6.83-6.89 (m, 1H).

### EXAMPLE 45

### Preparation of 2-[4-(2-ethylbutoxy)-3,5-dimethyl-1H-pyrazol-1-yl]ethylamine Trifluoroacetic Acid Salt

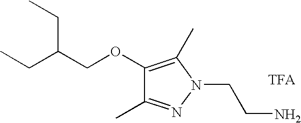

To a solution of Example 44 (70 mg, 0.20 mmol) in CH₂Cl₂ (2 mL) was added trifluoroacetic acid (2 mL). The resulting solution was stirred for 2 h at rt and then concentrated to an oil which was dried under vacuum to yield a waxy residue (63 mg, 90%). ESLC-MS m/z=240 (MH⁺); ¹H NMR (DMSO-d₆) δ 0.89 (t, J=7.1 Hz, 6H), 1.30-1.54 (m, 5H), 2.06 (s, 3H), 2.12 (s, 3H), 3.10-3.19 (m, 2H), 3.63 (d, J=4.8 Hz, 2H), 4.03-4.10 (m, 2H), 7.83 (s, 3H).

### EXAMPLE 46

### Preparation of 4-(4-chlorophenoxy)-3,5-dimethyl-1H-pyrazole

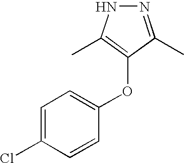

To a solution of 3-(4-chlorophenoxy)-2,4-pentanedione (1.0 g, 4.41 mmol) and hydrazine monohydrate (0.38 mL, 6.62 mmol) in 22 mL of ethanol was added a drop of acetic acid. The mixture was heated at reflux for 2 h. The reaction mixture was concentrated in vacuo to give a yellow solid. GC/MS m/z: 222 (M⁺).

### EXAMPLE 47

### Preparation of 2-[4-(4-chlorophenoxy)-3,5-dimethyl-1H-pyrazol-1-yl]ethylamine

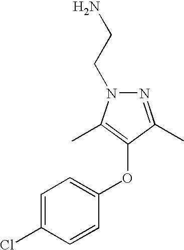

A mixture of the yellow solid obtained in Example 46, 2-chloroethylamine hydrochloride (0.77 g, 6.62 mmol), NaOH (0.71 g, 17.64 mmol), Bu₄NHSO₄ (0.06 g, 0.18 mmol), and CH₃CN (3 mL) was heated at reflux for 3 h. The reaction mixture was cooled to rt and diluted with water and EtOAc. The layers were separated and the aqueous was extracted with EtOAc twice. The combined organic layer was dried (Na₂SO₄) and concentrated down under reduced pressure. The crude product was purified by flash column chromatography eluting with MeOH/EtOAc (20:80) to yield the product as a thick oil (0.63 g, 53%): ¹H NMR (CDCl₃) δ 7.23-7.19 (m, 2H), 6.81-6.78 (m, 2H), 4.12-4.08 (m, 2H), 3.31-3.25 (m, 4H), 2.10 (s, 3H), 2.04 (s, 3H); GC/MS m/z (%): 265 (M⁺, 22.6), 235 (100).

### EXAMPLE 48

### Preparation of 3,5-dimethyl-4-nitro-1H-pyrazole

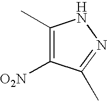

To a −15° C. solution of 3,5-dimethylpyrazole (480 mg, 5 mmol) in H₂SO₄ (10 mL) was added conc. HNO₃ (0.9 mL) dropwise. The resulting solution was stirred at −15° C. for 30 min and then let come to rt with stirring for 18 h. The reaction was poured into 100 mL of ice water and the resulting solution extracted with CH₂Cl₂. The organics were dried (MgSO₄) and concentrated to yield a white solid (480 mg, 68%). R_{f}=0.60 (EtOAc); ESLC-MS m/z=142 (MH⁺); ¹H NMR (DMSO-d₆) δ 2.27 (s, 6H); {¹H}¹³C NMR (DMSO-d₆) δ 144.1, 130.6, 13.5.

### EXAMPLE 49

### Preparation of Tert-Butyl 2-(3,5-dimethyl-4-nitro-1H-pyrazol-1-yl)ethylcarbamate

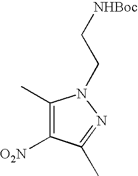

To a solution of 3,5-dimethyl-4-nitropyrazole (28.8 g, 0.204 mol) in DMF (500 mL) was added Cs₂CO₃ (133 g, 0.408 mol) and N-Boc-2-bromoethylamine (55 g, 0.245 mol).

The resulting solution was head to 60° C. for 18 h, cooled to rt, and poured into water to produce a precipitate which was collected by filtration, washed with water, and dried in a vacuum oven to yield a white solid (21.5 g, 37%). R_{f}=0.28 (50:50 EtOAc:hex (v/v)); ESLC-MS m/z=285 (MH⁺); ¹H NMR (DMSO-d₆) δ 6.93 (t, J=5.7 Hz, 1H), 4.11-4.04 (m, 2H), 3.27-3.16 (m, 2H), 2.52 (s, 3H), 2.36 (s, 3H), 1.31 (s, 9H).

### EXAMPLE 50

### Preparation of Tert-Butyl 2-(4-amino-3,5-dimethyl-1H-pyrazol-1-yl)ethylcarbamate

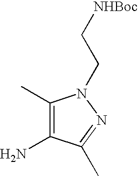

An argon-flushed Parr bottle was charged with Example 49 (21.5 g, 75.7 mmol), 10% Pd on carbon-degussa type, EtOAc (100 mL), and EtOH (100 mL). The resulting mixture was put under a hydrogen atmosphere (60 psi) and shaken for 5 days. The reaction was put back under argon, filtered through Celite®, and the filtrate concentrated to yield a white solid (14.9 g, 77%). R_{f}=0.30 (90:10 EtOAc:MeOH (v/v)); ESLC-MS rr/z=255 (MH⁺); ¹H NMR (DMSO-d₆) & 6.84 (t, J=5.7 Hz, 1H), 3.86-3.78 (m, 2H), 3.31 (s, 2H), 3.15-3.05 (m, 2H), 2.03 (s, 3H), 1.97 (s, 3H), 1.37 (s, 9H).

### EXAMPLE 51

### Preparation of Tert-Butyl 2-{4-[(isopropylsulfonyl)amino]-3,5-dimethyl-1H-pyrazol-1-yl}ethylcarbamate

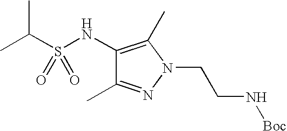

To a vial containing a solution of the starting aniline (75 mg, 0.29 mmol) in THF (1 mL) and polystyrylpyridine (100 mg) was added isopropylsulfonyl chloride (0.090 mL). The resulting mixture was agitated for 18 h at 60° C., cooled to rt, diluted with MeOH (1 mL) and filtered. The filtrate was concentrated and the residue purified by HPLC. ESLC-MS m/z=361 (MH⁺).

### EXAMPLE 52

### Preparation of 1-(2-aminoethyl)-N-isopropyl-3,5-dimethyl-1H-pyrazol-4-amine

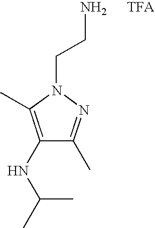

To a 8 mL vial charged with a solution of tert-butyl 2-(4-amino-3,5-dimethyl-1H-pyazol-1-yl)ethylcarbamate (75.1 mg, 0.295 mmol) in 3 mL of 1,2-dichloroethane was added acetone (0.0217 mL, 0.295 mmol) and NaB(OAc)₃H (87.6 mg, 0.413 mmol). The suspension was shaken on a shaker for 3 d. Saturated NaHCO₃ aqueous solution (1.5 mL) was added and the mixture was shaken overnight. The layers were separated. The organic layer was concentrated under reduced pressure. The crude product was purified on HPLC.

A solution of the purified product in CH₂Cl₂ (1 mL) was treated with trifluoroacetic acid (1 mL) for 3 h. Evaporation afforded a solid (56.7 mg, 62%): ¹H NMR (CDCl₃) δ 7.94 (b, 2H), 4.19-4.5 (m, 2H), 3.44-3.39 (m, 1H), 3.23-3.17 (m, 2H), 2.29 (s, 3H), 2.19 (s, 3H), 1.23 (d, J=6.7 Hz, 6H); LC/MS m/z: 197 (M+1, 100).

### EXAMPLE 53

### Preparation of Tert-Butyl 2-[3,5-dimethyl-4-(methylamino)-1H-pyrazol-1-yl]ethylcarbamate

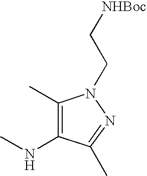

To a solution of the amine from Example 50 (9.0 g, 35 mmol) in MeOH (100 mL) was added NaOMe (40 mL of a 25 wt % solution in MeOH) and paraformaldehyde (1.4 mg, 48 mmol). The resulting solution was stirred for 18 h at rt. To the reaction was then added NaBH₄ (1.6 mg, 43 mmol) and the mixture was heated to reflux for 1 h. The reaction was then cooled to rt and a 1.0 M solution of NaOH in water was added followed by a saturated aqueous solution of NaCl. The mixture was stirred for 15 min and then extracted with EtOAc. The combined organics were dried (MgSO₄) and concentrated to a red oil which was purified on silica with a gradient eluant of 100% EtOAc to 9:1 EtOAc:MeOH (v/v) to yield an orange oil which crystallized on standing (9.28 g, 99%). R_{f}=0.30 (90:10 EtOAc:MeOH (v/v)); ESLC-MS m/z=269 (MH⁺); ¹H NMR (DMSO-d₆) δ 6.85 (t, J=5.7 Hz, 1H), 3.90-3.80 (m, 2H), 3.75 (s, 1H), 3.18-3.08 (m, 2H), 2.48 (s, 3H), 2.07 (s, 3H), 2.00 (s, 3H), 1.35 (s, 9H).

### EXAMPLE 54

### Preparation of Benzyl 4-(1-acetyl-2-oxopropyl)-1-piperazinecarboxylate

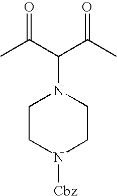

A solution of benzylpiperazine carboxylate (1.9 mL, 10 mmol) in DMF (5 mL) was stirred at rt under an argon atmosphere. Neat 3-chloro-2,4-pentanedione (0.36 mL, 3 mmol) was added and the resulting solution stirred 18 h at rt. The reaction was poured into H₂O (50 mL) and the resulting mixture extracted with EtOAc. The combined organics were washed with saturated aqueous NaCl, dried (MgSO₄) and concentrated. The residue was filtered through silica__n EtOAc and the filtrate reduced to 1.40 g of a yellow oil which was used without further purification. R_{f}=0.47 (50:50 EtOAc:hex (v/v)); ESLC-MS m/z=319 (MH⁺).

### EXAMPLE 55

### Preparation of Benzyl 4-(1-[2-[(tert-butoxycarbonyl)amino]ethyl]-3,5-dimethyl-1H-pyrazol-4-yl)-1-piperazinecarboxylate

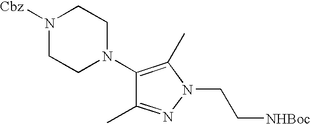

Example 54 (1.4 g) was dissolved in EtOH (7 mL) and stirred while a solution of N-boc-2-hydrazidoethylamine (1.5 g, 8.8 mmol) in EtOH (7 mL) was added. The resulting solution was stirred 18 h at rt. The reaction was concentrated to an oil and the oil partitioned between water and EtOAc. The organic layer was collected and the aqueous layer extracted with EtOAc. The combined organics were dried (MgSO₄), concentrated, and the resulting residue purified on silica in EtOAc to yield a white solid (1.40 g, 100% for two steps). R_{f}=0.52 (EtOAc); ESLC-MS m/z=458 (MH⁺); ¹H NMR (DMSO-d₆) δ 7.40-7.26 (m, 5H), 6.89 (t, J=5.7 Hz, 1H), 5.08 (s, 2H), 3.88-3.83 (m, 2H), 3.48-3.40 (m, 4H), 3.17-3.10 (m, 2H), 2.83-2.80 (m, 4H), 2.10 (s, 3H), 2.07 (s, 3H), 1.35 (m, 9H).

### EXAMPLE 56

### Preparation of Tert-Butyl 2-[3,5-dimethyl-4-(1-piperazinyl)-1H-pyrazol-1-yl]ethylcarbamate

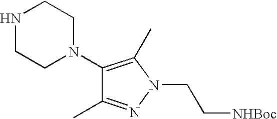

Example 55 (1.40 g, 3.0 mmol) was dissolved in a mixture of EtOH (10 mL) and EtOAc (10 mL) and added to 10% Pd on carbon (250 mg). The resulting mixture was stirred under an atmosphere of hydrogen for 3 days. The reaction was put under argon, filtered through celite. And the filtrate concentrated to a white solid (0.92 g, 85%). ESLC-MS m/z=324 (MH⁺); ¹H NMR (DMSO-d₆) δ6.89 (t, J=5.6 Hz), 3.88-3.82 (m, 2H), 3.18-3.10 (m, 2H), 2.80-2.70 (m, 8H), 2.10 (s, 3H), 2.09 (2, 3H), 1.35 (s, 9H).

### EXAMPLE 57

### Preparation of Tert-Butyl 2-(4-{4-[(isopropylamino)carbonyl]-1-piperazinyl}-3,5-dimethyl-1H-pyrazol-1-yl)ethylcarbamate

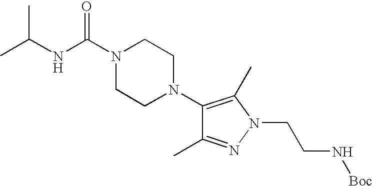

To a solution of Example 59 (50 mg, 0.15 mmol) in DMF (0.5 mL) was added isopropylisocyanate (0.030 mL). The reaction was agitated at 60° C. for 18 h, cooled to rt and quenched by the addition of MeOH. The product was purified by HLPC. ESLC-MS m/z=409 (MH⁺).

### EXAMPLE 58

### Preparation of 2-[4-(4-acetyl-1-piperazinyl)-3,5-dimethyl-1H-pyrazol-1-yl]ethylamine Bis Trifluoroacetic Acid Salt

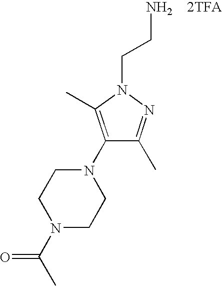

To a 8 mL vial charged with piperidinomethyl polystyrene (248.2 mg, 3.57 mmol/g, 0.886 mmol) was added a solution of tert-butyl 2-[3,5-dimethyl-4-(1-piperazinyl)-1H-pyazol-1-yl]ethylcarbamate (95.5 mg, 0.295 mmol) in 4 mL of CH₂Cl₂ and acetic acid chloride (0.021 mL, 0.295 mmol). The mixture was shaken on a shaker overnight and the solid was filtered off. The filtrate was concentrated under reduced pressure. The residue was purified on HPLC to yield a solid.

To a solution of the solid in CH₂Cl₂ (1 mL) was added trifluoroacetic acid (1 mL). The mixture was shaken on a shaker for 3 h. The volatiles were removed under reduced pressure to yield a solid (116.4 mg, 80%). ¹H NMR (CDCl₃) δ 7.87 (b, 2H), 4.09-4.04 (m, 2H), 3.511-3.3.44 (m, 4H), 3.18-3.12 (m, 2H), 2.89-2.79 (m, 4H), 2.15 (s, 3H), 2.12 (s, 3H), 2.01 (s, 3H); LC/MS m/z(%): 266 (M+1, 100).

### EXAMPLE 59

### Preparation of 2-[4-(4-acetyl-1-piperazinyl)-3,5-dimethyl-1H-pyrazol-1-yl]ethylamine

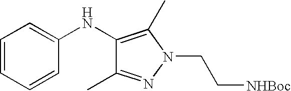

A solution of the Example 50 (150 mg, 0.59 mmol), phenylboronic acid (144 mg, 1.18 mmol), triethylamine (164 μL, 1.18 mmol) and copper(II) acetate (110 mg, 0.59 mmol) was stirred in CH₂Cl₂ (5 mL) at rt for 18 h. The reaction was diluted with CH₂Cl₂ and washed with saturated aqueous ammonium chloride. The organic layer was collected, dried (MgSO₄), adsorbed onto silica, and purified on silica using a gradient of 1:1 to 4:1 EtOAc:hex (v/v) as eluant to yield, after concentration, a light colored solid (133 mg, 68%). R_{f}=0.51 (EtOAc); ESLC-MS m/z=331 (MH⁺); ¹H NMR (DMSO-d₆) δ 1.36 (s, 9H), 1.91 (s, 3H), 2.00 (s 3H) 3.16-3.25 (m, 2H), 3.92-4.00 (m, 2H), 6.41-6.47 (m, 2H), 6.48-6.55 (m, 1H), 6.87 (s, 1H), 6.88-6.94 (m, 1H), 6.97-7.05 (m, 2H).

### EXAMPLE 60

### Preparation of 1-(2-aminoethyl)-3,5-dimethyl-N-phenyl-1H-pyrazol-4-amine Dihydrochloride

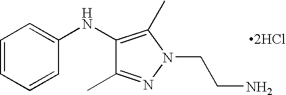

To a solution of Example 59 (130 mg, 0.40 mmol) was added a 4.0 N solution of HCl in 1,4-dioxane (5 mL). The resulting solution was stirred for 4 h at rt and then concentrated to a foamy solid which was dried under vacuum to yield a light purple solid (130 mg, 100%). ESLC-MS m/z=231 (MH⁺); ¹H NMR (DMSO-d₆) δ 1.95 (s, 3H), 2.05 (s, 3H), 3.16-3.25 (m, 2H), 4.15-4.21 (m, 2H), 6.45-6.50 (m, 2H), 6.50-6.57 (m, 1H), 6.99-7.07 (m, 2H), 8.06 (s, 3H).

### EXAMPLE 61

### Preparation of Tert-Butyl 2-{3,5-dimethyl-4-[methyl(phenyl)amino]-1H-pyrazol-1-yl}ethylcarbamate

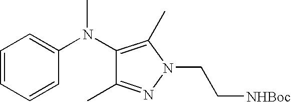

A mixture of Example 50 (440 mg, 1.64 mmol), phenylboronic acid (400 mg, 3.3 mmol), triethylamine (0.45 mL, 3.3 mmol), and copper(II) acetate (300 mg, 1.64 mmol) was stirred in CH₂Cl₂ (15 mL) for 18 h at rt. The reaction was diluted with CH₂Cl₂ and washed with a saturated aqueous solution of ammonium chloride. The organic layer was collected, dried (MgSO₄), and concentrated to an oil which was purified on silica using 2:1 EtOAc:hexane (v/v) as an eluant, to yield, after concentration, a solid (139 mg, 25%). R_{f}=0.72 (EtOAc); ESLC-MS m/z=345 (MH⁺); ¹H NMR (DMSO-d₆) δ 1.35 (s, 9H), 1.86 (s, 3H), 1.97 (s, 3H), 3.08 (s, 3H), 3.18-3.27 (m, 2H), 3.93-4.00 (m, 2H), 6.48-6.54 (m, 2H), 6.56-6.63 (m, 1H), 6.89-6.96 (m, 1H), 7.05-7.12 (m, 2H).

### EXAMPLE 62

### Preparation of 1-(2-aminoethyl)-N,3,5-trimethyl-N-phenyl-1H-pyrazol-4-amine

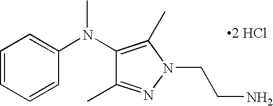

Example 61 (130 mg, 0.40 mmol) was stirred in a 4.0 N HCl in 1,4-dioxane (2.5 mL) for 3 h at rt. The reaction was concentrated to an oil, washed with Et₂O and dried under vacuum to yield a solid (109 mg, 86%). ¹H NMR (DMSO-d₆) δ 1.88 (s, 3H), 2.03 (s, 3H), 3.10 (s, 3H), 3.17-3.26 (m, 2H), 4.17-4.24 (m, 2H), 6.50-6.56 (m, 2H), 6.58-6.65 (m, 1H), 7.07-7.15 (m, 2H), 8.17 (s, 3H).

Examples 63-187 listed in the Tables 1 and 2 below were synthesized by the preparative methods described above or by using other known synthetic techniques in the art examples of which include those described by Schofield et al., Heteroaromatic Nitrogen Compounds: The Azoles, published by Cambridge University Press, (1976); and “Five Membered Heterocycles with Two Heteroatoms” from section 3 (1,2-Azoles), Chapter 4 of Heterocyclic Chemistry II—Five Membered Heterocycles, ed. by Gupta et al., publ. by Springer-Verlag, pages 435-454, (1999), each of which is incorporated in its entirety by reference.

Table 1 shows various embodiments of the described compounds.

Table 2 show various embodiment of the described compounds when R═R′-phenyl-CH₂.

TABLE 1

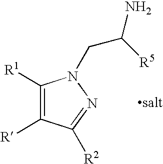

| | | | | | HPLC RT | Mass Spec | |
|---|---|---|---|---|---|---|---|
| Example | R′ | R¹ | R² | R⁵ | (min)* | [source] | salt |
| 63 | Me | Me | Me | H | 3.5 | 154.2 | HCl |
| 64 | n-Bu | Me | Me | H | 3.23 | 210.3 | HCl |
| 65 | 3-butenyl | Me | Me | H | 2.67 | 208 | |
| 66 | n-Bu | Me | Me | H | 3.23 | 210.3 | TFA |
| 67 | Et | Me | Me | H | 2.57 | 182.1(M + H) | TFA |
| 68 | n-Pr | Me | Me | H | 2.74 | 196.1(M + H) | TFA |
| 69 | n-Pent | Me | Me | H | 2.95 | 224.1(M + H) | TFA |
| 70 | Et | Me | Me | H | 2.42 | 168.1(M + H) | TFA |
| 71 | 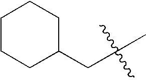 | Me | Me | H | 3.06 | 236.2(M + H) | TFA |
| 72 | i-BLi | Me | Me | H | 2.79 | 196.1(M + H) | TFA |
| 73 | 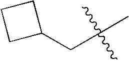 | Me | Me | H | 2.85 | 208.1(M + H) | TFA |
| 74 | 2-propenyl | Me | Me | H | 2.61 | 194(M + H) | TFA |
| 75 | 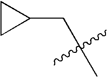 | Me | Me | H | 2.63 | 194.1(M + H) | TFA |
| 76 | i-Pr | Me | Me | H | 2.68 | 182.1(M + H) | TFA |
| 77 | vinyl | Me | Me | H | 2.6 | 180.0(M + H) | TFA |
| 78 | 3-F-Pr | Me | Me | H | 2.57 | 214.1(M + H) | TFA |
| 79 | 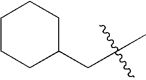 | Et | Et | H | 3.55 | 264.3(M + H) | |
| 80 | Et | Et | Et | H | 2.83 | 210.1(M + H) | |
| 81 | n-Pr | Et | Et | H | 2.83 | 224.2(M + H) | |
| 82 | Et | Et | Et | (S)-Me | 2.58 | 196.1 | |
| 83 | 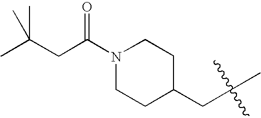 | Me | Me | (S)-Me | 2.82 | 349 | TFA |
| 84 | 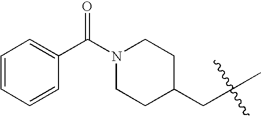 | Me | Me | (S)-Me | 2.73 | 354 | TFA |
| 85 | 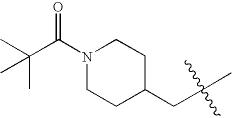 | Me | Me | (S)-Me | 2.75 | 335 | TFA |
| 86 | 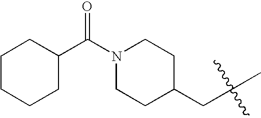 | Me | Me | (S)-Me | 2.84 | 361 | TFA |

TABLE 2

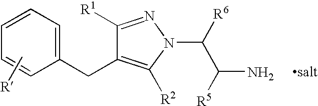

| | | | | | | HPLC | | |
|---|---|---|---|---|---|---|---|---|
| | | | | | | RT | | |
| Ex. | R′ | R¹ | R² | R⁵ | R⁶ | (min)* | Mass Spec | Salt |
| 87 | H | Et | Et | H | H | 4.34 | 258 | |
| 88 | 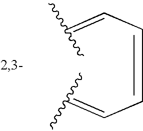 | Et | Et | H | H | 3.59 | 308.3(M + H) | HCl |
| 89 | 2-Ph | Et | Et | H | H | 3.70 | 334.3(M + H) | HCl |
| 90 | 4-tBu | Et | Et | H | H | 3.71 | 314.3(M + H) | HCl |
| 91 | 4-Ph | Et | Et | H | H | 3.66 | 334.3(M + H) | HCl |
| 92 | 3-OPh | Et | Et | H | H | 3.48 | 350.3(M + H) | HCl |
| 93 | 4-F | Et | Et | H | H | 2.91 | 276.3(M + H) | HCl |
| 94 | 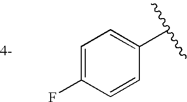 | Et | Et | H | H | 3.40 | 352.3(M + H) | HCl |
| 95 | 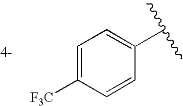 | Et | Et | H | H | 3.80 | 402.3(M + H) | HCl |
| 96 | 4-NO₂ | Et | Et | H | H | | 303 | |
| 97 | 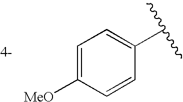 | Et | Et | H | H | 3.58 | 364.3(M + H) | FA |
| 98 | 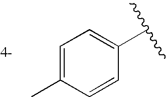 | Et | Et | H | H | 3.77 | 348.3(M + H) | FA |
| 99 | 4-Br | Et | Et | H | H | 3.55 | 338.2(M + 2) | FA |
| 100 | 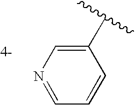 | Et | Et | H | H | 2.15 | 335.3(M + H) | FA |
| 101 | 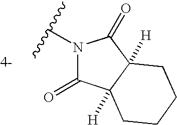 | Et | Et | H | H | 2.97 | 409(MH+) | TFA |
| 102 | 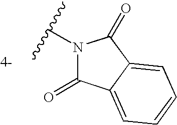 | Et | Et | H | H | 3.03 | 403(MH+) | TFA |
| 103 | 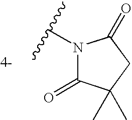 | Et | Et | H | H | 2.81 | 383(M + H)⁺[electrospray] | TFA |
| 104 | 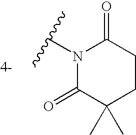 | Et | Et | H | H | 2.01 | 397 | HCl |
| 105 | 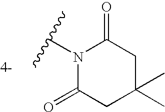 | Et | Et | H | H | 2.05 | 397 | HCl |
| 106 | 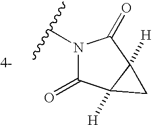 | Et | Et | H | H | 2.66 | 367 | HCl |
| 107 | 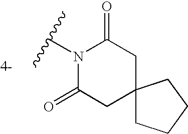 | Et | Et | H | H | 2.99 | 423 | HCl |
| 108 | 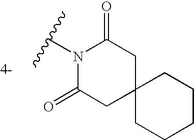 | Et | Et | H | H | 3.11 | 437 | HCl |
| 109 | 4-Br | Et | Et | Me | Me | 4.07 | 364(M + H)+ | TFA |
| | | | | | | | [electrospray] | |
| 110 | 4-Ph | Et | Et | Me | Me | 4.51 | 362(M + H)+ | TFA |
| | | | | | | | [electrospray] | |
| 111 | 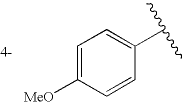 | Et | Et | Me | Me | 4.19 | 392(M + H)+[electrospray] | TFA |
| 112 | 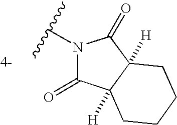 | Et | Et | (S)-Me | H | 3.05 | 423(MH+) | TFA |
| 113 | 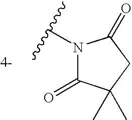 | Et | Et | (S)-Me | H | 2.90 | 397(M + H)+[electrospray] | HCl |
| 114 | 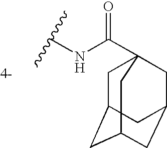 | Et | Et | (S)-Me | H | 3.30 | 449 | HCl |
| 115 | 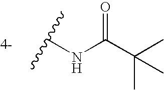 | Et | Et | (S)-Me | H | 2.97 | 371(MH+) | HCl |
| 116 | 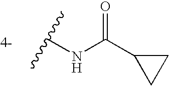 | Et | Et | (S)-Me | H | 2.77 | 355(MH+) | HCl |
| 117 | 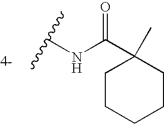 | Et | Et | (S)-Me | H | 3.23 | 411(MH+) | HCl |
| 118 | 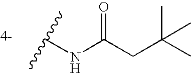 | Et | Et | (S)-Me | H | 3.06 | 385(MH+) | HCl |
| 119 | 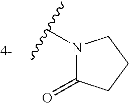 | Et | Et | (S)-Me | H | 2.79 | 355 | HCl |
| 120 | 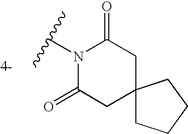 | Et | Et | (S)-Me | H | 3.07 | 437(MH+) | HCl |
| 121 | 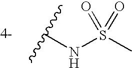 | Et | Et | (S)-Me | H | 2.69 | 365(MH+) | HCl |
| 122 | 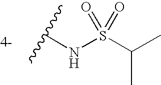 | Et | Et | (S)-Me | H | 2.83 | 393(MH+) | HCl |
| 123 | 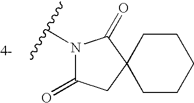 | Et | Et | (S)-Me | H | 2.96 | 424(MH+) | HCl |
| 124 | 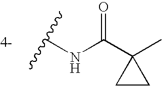 | Et | Et | (S)-Me | H | 2.94 | 369(MH+) | HCl |
| 125 | 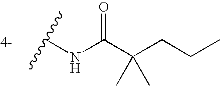 | Et | Et | (S)-Me | H | 3.10 | 399(MH+) | HCl |
| 126 | 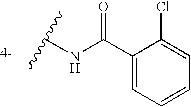 | Et | Et | (S)-Me | H | 2.26 | 425 | HCl |
| 127 | 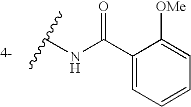 | Et | Et | (S)-Me | H | 2.37 | 421 | HCl |
| 128 | 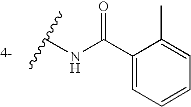 | Et | Et | (S)-ME | H | 2.26 | 405(M + H)⁺[eleotrospray] | HCl |
| 129 | 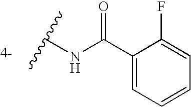 | Et | Et | (S)-Me | H | 2.26 | 409(M + H)⁺[eleotrospray] | HCl |
| 130 | 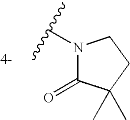 | Et | Et | (S)-Me | H | 2.21 | 383 | HCl |
| 131 | 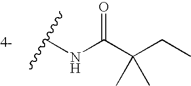 | Et | Et | (S)-Me | H | 2.29 | 385(MH+) | HCl |
| 132 | 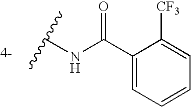 | Et | Et | (S)-Me | H | 2.32 | 459(M + H)⁺[electrospray] | HCl |
| 133 | 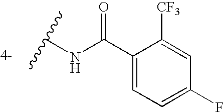 | Et | Et | (S)-Me | H | 2.34 | 477(MH+) | HCl |
| 134 | 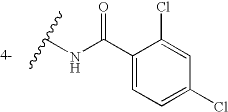 | Et | Et | (S)-Me | H | 2.41 | 459(MH+) | HCl |
| 135 | 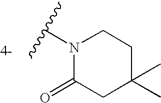 | Et | Et | (S)-Me | H | 2.17 | 397 | HCl |
| 136 | 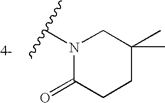 | Et | Et | (S)-Me | H | 2.21 | 397 | HCl |
| 137 | 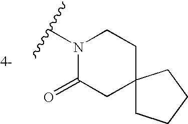 | Et | Et | (S)-Me | H | 2.38 | 423(MH+) | HCl |
| 138 | 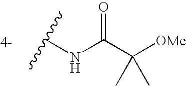 | Et | Et | (Sd-Me | H | 2.20 | 387(MH+) | HCl |
| 139 | 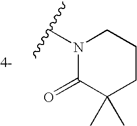 | Et | Et | (S)-Me | H | 2.23 | 397 | HCl |
| 140 | 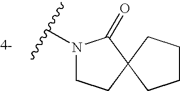 | Et | Et | (S)-Me | H | 2.48 | 409(MH+) | HCl |
| 141 | 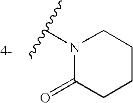 | Et | Et | (S)-Me | H | 2.00 | 369(MH+) | HCl |
| 142 | 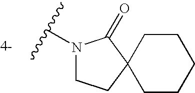 | Et | Et | (S)-Me | H | 2.50 | 423(MH+) | HCl |
| 143 | 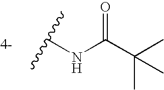 | Et | Et | (S)-Me | H | | | Mesylate |
| 144 | 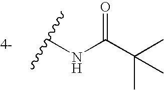 | Et | Et | (S)-Me | H | | | Tosylate |
| 145 | 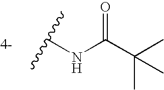 | Et | Et | (S)-Me | H | | | SA |
| 146 | 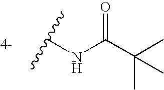 | Et | Et | (S)-Me | H | | | FA |
| 147 | 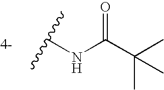 | Et | Et | (S)-Me | H | | | MA |
| 148 | 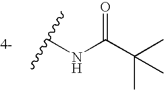 | Et | Et | (S)-Me | H | | | L-Tartaric |
| 149 | 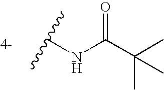 | Et | Et | (S)-Me | H | | | Citric A |
| 150 | 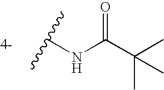 | Et | Et | (S)-Me | H | | | Malonic A |
| 151 | 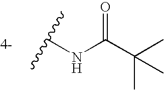 | Et | Et | (S)-Me | H | | | Oxalic A |
| 152 | 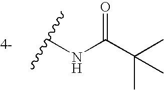 | Et | Et | (S)-Me | H | | | D-Tartaric |
| 153 | 4-Ph | Et | Et | Me | H | 3.79 | 348(M + H) [electrospray] | TFA |
| 154 | 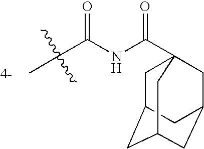 | Et | Et | (S)-Me | H | 2.91 | 484(M + H)+ | HCl |
| 155 | 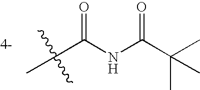 | Et | Et | (S)-Me | H | 2.44 | 405(M + H)+ | HCl |
| 156 | 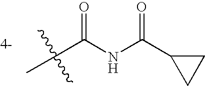 | Et | Et | (S)-Me | H | 2.09 | 389(M + H)+ | HCl |
| 157 | 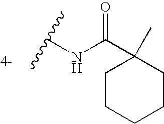 | Et | Et | (S)-Me | H | 2.62 | 445(M + H)+ | HCl |
| 158 | 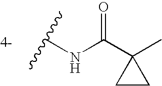 | Et | Et | (S)-Me | H | 2.33 | 403 | HCl |
| 159 | 4-F | CO₂Et | Me | H | H | | | maleic acid |
| 160 | 4-F | Et | CO₂Et | H | H | | | TFA |
| 161 | 4-NH₂ | Et | Et | (S)-Me | H | 0.76 | 287 | HCl |
| 162 | 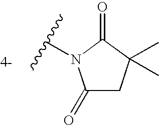 | Me | Me | H | H | 2.61 | 355(M + H+) | |
| 163 | 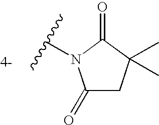 | Me | Me | Me | Me | 2.6 | 369(M + H)+ | |
| 164 | 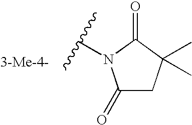 | Me | Me | H | H | 2.8 | 369(M + H)+ | TFA |
| 165 | 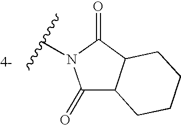 | Me | H | H | H | 2.8 | 381(M + H)+ | TFA |
| 166 | 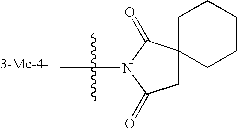 | Me | Me | Me | Me | 2.98 | 423(M + H)+ | HCl |
| 167 | 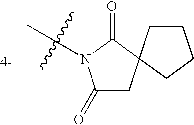 | Me | Me | Me | Me | 2.83 | 395(M + H)+ | HCl |
| 168 | 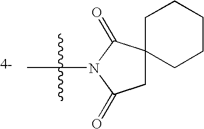 | Me | Me | Me | H | 2.94 | 409(M + H)+ | HCl |
| 169 | 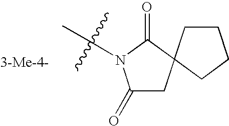 | Me | Me | Me | H | 2.95 | 409(M + H)+ | HCl |
| 170 | 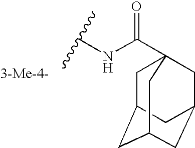 | Me | Me | H | H | 3.22 | 421(M + H)+ | TFA |
| 171 | 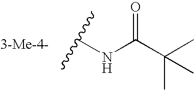 | Me | Me | H | H | 2.85 | 343(M + H)+ | TFA |
| 172 | 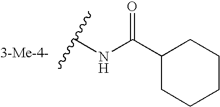 | Me | Me | H | H | 2.98 | 369(M + H)+ | TFA |
| 173 | 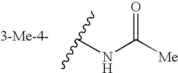 | Me | Me | H | H | 2.57 | 301(M + H)+ | TFA |
| 174 | 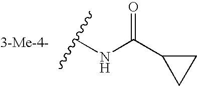 | Me | Me | H | H | 2.69 | 327(M + H)+ | TFA |
| 175 | 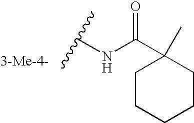 | Me | Me | H | H | 3.07 | 383(M + H)+ | TFA |
| 176 | 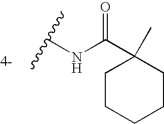 | Me | Me | H | H | 3.02 | 369(M + H)+ | TFA |
| 177 | 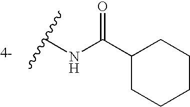 | Me | Me | H | H | 2.93 | 355(M + H)+ | TFA |
| 178 | 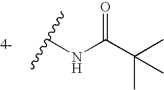 | Me | Me | H | H | 2.80 | 329(M + H)+ | TFA |
| 179 | 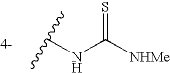 | Me | Me | H | H | 2.56 | 318(M + H)+ | TFA |
| 180 | 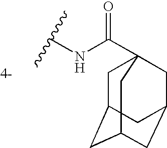 | Me | Me | H | H | 3.03 | 407(M + H)+ | TFA |
| 181 | 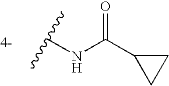 | Me | Me | H | H | 2.70 | 313(M + H)+ | TFA |
| 182 | 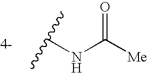 | Me | Me | H | H | 2.55 | 287(M + H)+ | TFA |
| 183 | 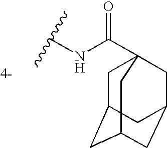 | Me | Me | Me | H | 3.12 | 421(M + H)+ | TFA |
| 184 | 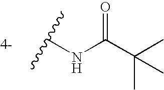 | Me | Me | Me | H | 2.77 | 343(M + H)+ | TFA |
| 185 | 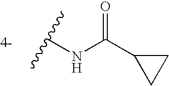 | Me | Me | Me | H | 2.62 | 327(M + H)+ | TFA |
| 186 | 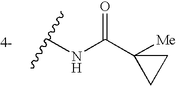 | Me | Me | Me | H | 2.72 | 341(M + H)+ | TFA |

Description of Method of Use

The compounds of Formula (I) and (II) interact with the 5-HT_{2C} receptor and are used in the treatment or prevention of diseases and/or behaviors that involve the 5-HT_{2C} receptor. These diseases and/or behaviors include obesity, obesity related disorders such as diabetes, feeding behavior, eating disorders such as bulimia, anorexia nervosa and premenstrual tension.

Further diseases and/or behaviors which can be treated or prevented include central nervous disorders, depressions, anxiety disorders, obsessive-compulsive disorders, sleep disorders, sexual dysfunction, psychoses, migraine, schizophrenia, drug or alcohol addiction and chronic fatigue syndrome.

Obesity is considered a major medical problem largely because it is a factor for a number of other diseases, and obese individuals have a higher chance of dying at a younger age than their leaner counterparts. Obesity is correlated with a much higher incidence of Type II diabetes (NIDDM), hypertension, hyperlipidemia, myocardial infarction, cancers, gallbladder disease, respiratory disease, gout, arthritis, and dermatological disease.

Targeting the 5-HT_{2C} receptor as method of treating obesity has previously been described (J. Pharmacology, 141, 429-435, (1987) and Psychopharmacology, 96, 93-100, (1988) each of which is hereby incorporated by reference). Agonists that are selective for this receptor would be expected to have superior properties with respect to other known appetite suppressants, such as serotonin/noradrenaline re-uptake inhibitors, which can lead to hypertension and/or cardiac valve defects.

Serotonin has been implicated in the regulation of feeding behavior and the infusion of 5-HT into the brain, resulting in lower food intake by promoting satiety. Furthermore, drugs which increase the concentration of 5-HT in the synaptic cleft by increasing 5-HT release and/or inhibiting re-uptake of the transmitter (such as Redux® (dexfenfluramine) and sibutramine) are effective long term treatments for obesity. However, while activation of several (5-HT_{1A}, 5-HT_{1B}, 5-HT_{2A}, and 5-HT_{2C}) subtypes of 5-HT receptors has been demonstrated to elicit effects on food intake, the best data available to date suggests that 5-HT_{2C} receptor agonists produce a decrease in food intake which is associated with the least likely potential for side effects, 5-HT_{2C} receptors are localized to the hypothalamus and the brainstem, two brain regions known to play a critical role in the modulation of food intake.

Serotonin produces physiological effects by acting on a heterogeneous family of receptors. The lack of selective agonists and antagonists for all of the individual subtypes of serotonin receptors has prevented a complete characterization of the physiological role of each receptor subtype.

Activation of both 5-HT_{2A} and 5-HT_{2C} receptors decrease food intake. However, while the 5-HT_{2C} receptor has been implicated in the regulation of satiety, 5-HT_{2A} receptor agonists are thought to decrease food intake by disrupting the ability of the animal to feed. Non-selective agonists/partial agonists (mCPP, TFMPP) at the 5-HT_{2C} receptor have been shown to reduce food intake in rats and to accelerate the appearance of the behavioral satiety sequence. Importantly, the hypophagic effects of mCPP are antagonized by the highly selective (at least 100-fold selective) 5-HT_{2C} receptor antagonist SB-242084. Recent findings from studies in normal human volunteers and obese subjects administered mCPP have also shown decreases in food intake. Thus, a single injection of mCPP decreased food intake in female volunteers and subchronic treatment for a 14 day period decreased the appetite and body weight of obese male and female subjects.

Although mCPP is a non-selective 5-HT agonist, the observations that the anorectic action of the drug is:
(a) absent in 5-HT_{2C} knockout mice; and
(b) antagonized by the 5-HT_{2C} receptor antagonist SB-242084 in rats,
   suggests that it decreases food intake via an agonist action at the 5-HT_{2C} receptor. Therefore, both animal and human data strongly implicate the involvement of the 5-HT_{2C} receptor in satiety.

Antagonist studies have shown that the selective 5-HT_{2C} receptor antagonist SB-242084 is highly effective in reversing the hypophagic actions of dexfenfluramine in the rat. Furthermore, the 5-HT₂ receptor antagonist, ritanserin, reversed the anorectic effect of dexfenfluramine in human volunteers. As ritanserin has a 10,000-fold selectivity for the 5-HT₂ receptors (pKi 8.9) over 5-HT₁ receptors, a crucial role for the 5-HT₂ receptors in the anorectic action of dexfenfluramine in humans is suggested.

The importance of the 5-HT_{2C} receptor in mediating feeding behavior is further supported by studies on mutant 5-HT_{2C}-knockout mice lacking this receptor (Nature, 374, 542-546 9(1995) and British Journal of Pharmacology, 128, 113-209 (1999), which is hereby incorporated by reference). Interestingly, the knockout mice show significantly greater weight gain and adipose tissue deposits over time compared to wild-type mice. Additional studies have confirmed that 5-HT_{2C} knockout mice overeat and become obese which appears due to a defect in their satiety mechanism. In the behavioral satiety sequence model, knockout animals continued to eat for a significantly longer period of time than the wild-type controls. The prolonged eating in the 5-HT_{2C} receptor knockout mice was enhanced by access to a sweet diet, suggesting that the 5-HT_{2C} receptor may play a role in palatability.

It is significant that the decrease in food intake induced by dexfenfluramine is markedly attenuated in 5-HT_{2C} receptor knockout mice. These results suggest that dexfenfluramine enhances satiety and decreases food intake via an agonist action on 5-HT_{2C} receptors. In addition, in wild-type animals these anorectic effects of dexfenfluramine are blocked by the 5-HT_{2C}-selective antagonist SB-242084. These data are consistent with the clinical evidence that the anorectic effect of dexfenfluramine was blocked by the 5HT₂ receptor antagonist ritanserin.

Thus, anorectic activity of the compounds of Formula (I) and (II) can be determined by measurement of their binding affinity to the 5-HT_{2C} receptor. Other research groups have explored this approach and have disclosed a number of ligands for the 5-HT_{2C} receptor. (Cerebrus Pharmaceuticals: WO 00/12502, WO 00/12481, WO 00/12475, WO 00/12510, WO 00/12482; Hoffman-La Roche: US005292732, US005646173; Yamanouchi Pharmaceutical: WO98/56768; and Akzo Nobel: EP 0 863 136 A1, each of which is hereby incorporated by reference).

The following assay was performed to determine the effect of the compounds of formula (I) and (II) on the 5-HT_{2C} receptor:

AV-12 cell pellets expressing 5HT₂C, 5-HT_{2A} or 5-HT_{2B} receptors are homogenized in binding buffer (50 mM Tris-HCl, 10 mM MgCl₂, 10 uM pargyline, 0.1% Sodium Ascorbate, 0.5 mM EDTA, pH 7.4 using saturated Tris Base). Radioligand binding assays were performed as follows: 50 μl of various concentrations of test compound or reference compound (5-HT) are added to 50 μl of ¹²⁵I-DOI (1-(2,5-dimethoxy-4-iodophenyl)-2-aminopropane). Non-specific binding is defined by 10 uM 5-HT. The reaction is initiated by the addition of 100 μl membrane homogenate and incubated for 45 minutes at room temperature (23° C.). Bound radioactivity is determined after rapid filtration using a Brandel Cell Harvester. Filter plates (GF/B pretreated with 0.5% polyethyleneimine) are washed twice with ice-cold wash buffer (50 mM Tris-HCl, pH 7.4 using saturated Tris Base) and radioactivity determined using a Microbeta counter. Data (IC₅₀ values) are analyzed using a four parameter logistic equation (Graph Pad).

All example compounds of Formula 1 and 11 were tested in the above assays and were found to have an effect on 5-HT_{2C} at or below a concentration of 10 μM.

Other embodiments of the invention will be apparent to the skilled in the art from a consideration of this specification or practice of the invention disclosed herein. It is intended that the specification and examples be considered as exemplary only, with the scope and spirit of the invention being indicated by the following claims.

1. A compound of formula (I) or (II): 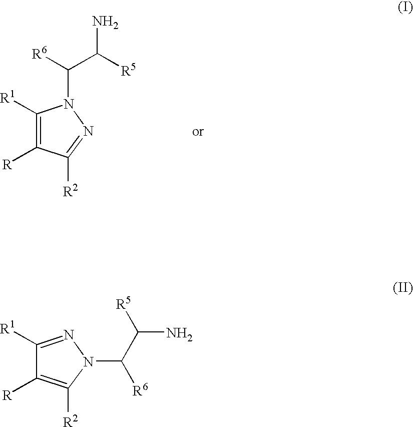
wherein:
R is selected from the group consisting of:
(a) hydrogen,
(b) (C₁-C₅)-alkyl optionally substituted with:
(b1) (C₃-C₈)cycloalkyl, or
(b2) a four to eight membered saturated or unsaturated heterocyclic ring which contains one to four heteroatoms selected from the group consisting of nitrogen, oxygen and sulfur, wherein said heterocyclic ring contains at least one carbon atom and wherein said heterocyclic ring is optionally substituted with one to two —C(═O)R₉,
(c) (C₁-C₅)-alkenyl optionally substituted with (C₁-C₅)-alkyl,
(d) (C₁-C₅)-alkynyl optionally substituted with (C₁-C₅)-alkyl,
(e) (C₆-C₁₀)-aryl optionally substituted with one to three substituents selected from the group consisting of:
(e1) halogen,
(e2) (C₁-C₅)-alkyl optionally substituted by halogen, and
(e3) (C₁-C₅)-alkoxy,
(f) (C₆-C₁₀)-aryl-(C₁-C₅)-alkyl wherein the aryl is optionally substituted with one to three substituents selected from the group consisting of:
(f1) halogen,
(f2) nitro,
(f3) (C₁-C₅)-alkyl optionally substituted by halogen,
(f4) (C₆-C₁₀)-aryl optionally substituted with one to three substituents selected from the group consisting of:
(f4a) halogen,
(f4b) (C₁-C₅)-alkyl optionally substituted with halogen, and
(f4c) (C₁-C₅)-alkoxy,
(f5) (C₆-C₁₀)-aryl-(C₁-C₅)-alkoxy,
(f6) a four to eight membered saturated or unsaturated heterocyclic ring which contains one to four heteroatoms selected from the group consisting of nitrogen, oxygen and sulfur wherein said heterocyclic ring contains at least one carbon atom wherein said heterocyclic ring is optionally substituted with one to four substituents selected from the group consisting of:
(f6a) oxo,
(f6b) (C₁-C₅)-alkyl optionally substituted with (C₃-C₈)-spiro-cycloalkyl ring,
(f6c) (C₆-C₁₀)-aryl,
(f6d) (C₃-C₈)-spiro-cycloalkyl ring,
(f6e) a bicyclo cycloalkyl ring wherein each ring is independently a five to six membered cycloalkyl ring,
(f6e) a tricyclo cycloalkyl ring wherein each ring is independently a five to six membered cycloalkyl ring,
(f6f) (C₁-C₅)-dialkyl,
(f7) a fused bicyclo ring wherein one ring is a four to eight membered saturated or unsaturated heterocyclic ring which contains one to four heteroatoms selected from the group consisting of nitrogen, oxygen and sulfur, wherein said heterocyclic ring contains at least one carbon atoms and said heterocyclic ring is optionally substituted with one to two oxo substituents, and the other ring is a saturated or unsaturated three to eight membered cycloalkyl ring,
(f8) NR³R⁴,
(f9) NR¹³C(═O)—R¹⁴
(f10) NR¹³S(═O)R¹⁵, and
(f11) C(═O)NR¹³C(═O)R¹⁶.
(g) a fused bicyclo ring wherein both rings are saturated or unsaturated five to six membered cycloalkyl rings, and
(h) a four to eight membered saturated or unsaturated heterocyclic ring which contains one to four heteroatoms selected from the group consisting of nitrogen, oxygen and sulfur, wherein said heterocyclic ring contains at least one carbon atom and wherein said heterocyclic ring is optionally substituted with one to four substituents selected from the group consisting of:
(h1) C(═O)R⁹,
(h2) C(═O)OR⁹,
(h3) C(═O)NR¹⁰R¹¹,
(h4) SO₂R¹²;
(i) NR³R⁴;
(j) NO₂, and
(j) OR⁸;
R¹ and R² are independently selected from the group consisting of
(a) hydrogen,
(b) (C₁-C₅)-alkyl optionally substituted with halogen,
(c) (C₂-C₅)-alkenyl optionally substituted with (C₁-C₅)-alkyl,
(d) (C₂-C₅)-alkynyl optionally substituted with (C₁-C₅)-alkyl,
(e) C(═O)R⁷,
(f) C(═O)OR⁷, and
(g) C(═O)NR³R⁴;
R³ and R⁴ are independently selected from the group consisting of
(a) hydrogen,
(b) (C₁-C₅)-alkyl optionally substituted with C₃-C₈-Cycloalkyl,
(c) (C₆-C₁₀)-aryl,
(d) (C₆-C₁₀)-aryl-C₁-C₅-alkyl optionally substituted with one to three halogens,
(e) C(═O)R⁹,
(f) C(═O)OR⁹,
(g) C(═O)NR¹⁰R¹¹,
(h) (C₃-C₈)-cycloalkyl, and
(i) —SO₂R¹²;
R⁵, R⁶ and R⁷ are independently selected from the group consisting of
(a) hydrogen, and
(b) (C₁-C₅)-alkyl;
R⁸ is selected from the group consisting of:
(a) hydrogen,
(b) (C₁-C₆)-alkyl optionally substituted with
(b1) halogen,
(b2) cyano, and
(b3) a four to eight membered saturated or unsaturated heterocyclic ring which contains one to four heteroatoms selected from the group consisting of nitrogen, oxygen and sulfur, wherein said heterocyclic ring contains at least one carbon atom and wherein said heterocyclic ring is optionally substituted with nitro,
(c) (C₂-C₅)-alkenyl optionally substituted with (C₁-C₅)-alkyl,
(d) (C₂-C₅)-alkynyl optionally substituted with (C₁-C₅)-alkyl,
(e) (C₆-C₁₀)-aryl optionally substituted by one to three halogens,
(f), (C₆-C₁₀)-aryl-C₁-C₅-alkyl wherein the (C₆-C₁₀)-aryl is optionally substituted with one to three substituents selected from the group consisting of cyano, halogen, (C₁-C₅)-alkoxy or phenyl,
(g) (C₆-C₁₀)-aryloxy-(C₁-C₅)-alkyl,
(h) (C₁-C₅)-alkyl-C(═O), and
(i) (C₆-C₁₀)-aryl-C(═O);
R⁹ is selected from the group consisting of:
(a) hydrogen,
(b) (C₁-C₅)alkyl optionally substituted with (C₃-C₈)-cycloalkyl,
(c) (C₆-C₁₀)-aryl optionally substituted by one to three substituents selected from the group consisting of:
(b1) halogen,
(b2) (C₁-C₅)-alkyl,
(b3) (C₁-C₅)-alkoxy, and
(b4) (C₃-C₈)-cycloalkyl,
(d) (C₆-C₁₀)-aryl-(C₁-C₅)-alkyl optionally substituted with halogen,
(e) a four to eight membered saturated or unsaturated heterocyclic ring which contains one to four heteroatoms selected from the group consisting of nitrogen, oxygen and sulfur, wherein said heterocyclic ring contains at least one carbon atom, and
(f) (C₃-C₈)-cycloalkyl;
R¹⁰ and R¹¹ are independently selected from the group consisting of:
(a) hydrogen,
(b) (C₁-C₅)-alkyl,
(c) (C₆-C₁₀)-aryl optionally substituted with one to three substituents selected from the group consisting of:
(c1) halogen,
(c2) (C₁-C₅)-alkyl, and
(c3) (C₁-C₅)-alkoxy,
(d) (C₆-C₁₀)aryl-(C₁-C₅)-alkyl wherein the (C₆-C₁₀)-aryl is optionally substituted with one to three substituents selected from the group consisting of:
(d1) halogen,
(d2) (C₁-C₅)-alkyl, and
(d3) (C₁-C₅alkoxy, and
(e) (C₃-C₈)cycloalkyl;
R¹² is selected from the group consisting of
(a) (C₁-C₅)-alkyl,
(b) (C₆-C₁₀)-aryl optionally substituted with one to three substituents selected from the group consisting of:
(b1) halogen,
(b2) (C₁-C₅)-alkyl, and
(b3) (C₁-C₅)-alkoxy, and
(c) C₆-C₁₀-aryl-(C₁-C₅)-alkyl wherein the C₆-C₁₀-aryl is optionally substituted with one to three substituents selected from the group consisting of:
(c1) halogen,
(c2) (C₁-C₅)-alkyl, and
(c3) (C₁-C₅)-alkoxy;
R¹³ is selected from the group consisting of hydrogen and (C₁-C₅)-alkyl;
R¹⁴ is selected from the group consisting of
(a) (C₁-C₅)-alkyl optionally substituted with substituents (C₁-C₅)-alkoxy, (C₃-C₈)-spiro-cycloalkyl, or (C₆-C₁₀)-aryl optionally substituted with one to three halogens,
(b) (C₆-C₁₀)-aryl optionally substituted with one to three substituents selected from the group consisting of halogen, (C₁-C₅)-alkoxy, and (C₁-C₅)-alkyl optionally substituted with halogen,
(c) (C₃-C₈)-cycloalkyl optionally substituted with one to three substituents selected from the group consisting of halogen, (C₁-C₅)-alkoxy, and (C₁-C₅)-alkyl optionally substituted with halogen, and
(d) a bicyclo cycloalkyl ring wherein each ring is independently a five to six membered cycloalkyl ring,
(e) a tricyclo cycloalkyl ring wherein each ring is independently a five to six membered cycloalkyl ring, and
(f) a four to eight membered saturated or unsaturated heterocyclic ring which contains one to four heteroatoms selected from the group consisting of nitrogen, oxygen and sulfur, wherein said heterocyclic ring contains at least one carbon atom and wherein said ring is optionally substituted with one to three substituents selected from the group consisting of halogen, (C₁-C₅)-alkoxy, and (C₁-C₅)-alkyl optionally substituted with halogen;
R¹⁵ is selected from the group consisting of
(a) hydrogen, and
(b) (C₁-C₅)-alkyl;
R¹⁶ is selected from the group consisting of
(a) hydrogen,
(b) (C₁-C₅)-alkyl,
(c) (C₃-C₈)-cycloalkyl,
(d) a bicyclo cycloalkyl ring wherein each ring is independently a five to six membered cycloalkyl ring, and
(e) a tricyclo cycloalkyl ring wherein each ring is independently a five to six membered cycloalkyl ring;
or a purified stereoisomer or stereoisomer mixture of said compound, or salt of said compound, stereoisomer or stereoisomer mixture.

2. The compound of claim 1 wherein:
R is selected from the group consisting of:
(a) (C₁-C₅)-alkyl optionally substituted with:
(a1) (C₃-C₈)-cycloalkyl, or
(a2) a four to eight membered saturated or unsaturated heterocyclic ring which contains one to four heteroatoms selected from the group consisting of nitrogen, oxygen and sulfur, wherein said heterocyclic ring contains at least one carbon atom and wherein said heterocyclic ring is optionally
(b) (C₁-C₅)-alkenyl optionally substituted with (C₁-C₅)-alkyl,
(c) (C₁-C₅)-alkynyl optionally substituted with (C₁-C₅)-alkyl, and
(d) (C₆-C₁₀)-aryl-(C₁-C₅)-alkyl wherein the aryl is optionally substituted with one to three substituents selected from the group consisting of:
(d1) halogen,
(d2) nitro,
(d3) (C₁-C₅)-alkyl optionally substituted by halogen,
(d4) (C₆-C₁₀)-aryl optionally substituted with one to three substituents selected from the group consisting of:
(d4a) halogen,
(d4b) (C₁-C₅)-alkyl optionally substituted with halogen, and
(d4c) (C₁-C₅)-alkoxy,
(d5) (C₆-C₁₀)-aryl-(C₁-C₅)-alkoxy,
(d6) a four to eight membered saturated or unsaturated heterocyclic ring which contains one to four heteroatoms selected from the group consisting of nitrogen, oxygen and sulfur wherein said heterocyclic ring contains at least one carbon atom wherein said heterocyclic ring is optionally substituted with one to four substituents selected from the group consisting of:
(d6a) oxo,
(d6b) (C₁-C₅)-alkyl optionally substituted with (C₃-C₈)-spiro-cycloalkyl ring,
(d6c) (C₆-C₁₀)-aryl,
(d6d) (C₃-C₈)-spiro-cycloalkyl ring,
(d6e) a bicyclo cycloalkyl ring wherein each ring is independently a five to six membered cycloalkyl ring,
(d6f) a tricyclo cycloalkyl ring wherein each ring is independently a five to six membered cycloalkyl ring, and
(d6g) (C₁-C₅)-dialkyl,
(d7) a fused bicyclo ring wherein one ring is a four to eight membered saturated or unsaturated heterocyclic ring which contains one to four heteroatoms selected from the group consisting of nitrogen, oxygen and sulfur, wherein said heterocyclic ring contains at least one carbon atoms and said heterocyclic ring is optionally substituted with one to two oxo substituents, and the other ring is a saturated or unsaturated three to eight membered cycloalkyl ring,
(d8) NR³R⁴,
(d9) NR¹³C(═O—R¹⁴
(d10) NR¹³S(═O)—R¹⁵, and
(d11) C(═O)NR³C(═O)R¹⁶;
R¹ and R² are independently selected from the group consisting of
(a) hydrogen,
(b) (C₁-C₅)-alkyl optionally substituted with halogen,
(c) (C₂-C₅)-alkenyl optionally substituted with (C₁-C₅)-alkyl, and
(d) (C₂-C₅)-alkynyl optionally substituted with (C₁-C₅)-alkyl;
R³ and R⁴ are independently selected from the group consisting of
(a) hydrogen,
(b) (C₁-C₅)-alkyl optionally substituted with C₃-C₈-cycloalkyl,
(c) (C₆-C₁₀)-aryl,
(d) (C₆-C₁₀)-aryl-C₁-C₅-alkyl optionally substituted with one to three halogens,
(e) C(═O)R⁹,
(f) C(═O)NR¹⁰R¹¹,
(g) (C₃-C₈)-cycloalkyl, and
(h) —SO₂R¹²;
R⁵ and R⁶ are independently selected from the group consisting of
(a) hydrogen, and
(b) (C₁-C₅)-alkyl;
R⁹ is selected from the group consisting of:
(a) hydrogen,
(b) (C₁-C₅)-alkyl optionally substituted with (C₃-C₈)-cycloalkyl,
(c) (C₆-C₁₀)-aryl optionally substituted by one to three substituents selected from the group consisting of:
(c1) halogen,
(c2) (C₁-C₅)-alkyl,
(c3) (C₁-C₅)-alkoxy, and
(c4) (C₃-C₈)cycloalkyl,
(d) (C₆-C₁₀)-aryl-(C₁-C₅)-alkyl optionally substituted with halogen,
(e) a four to eight membered saturated or unsaturated heterocyclic ring which contains one to four heteroatoms selected from the group consisting of nitrogen, oxygen and sulfur, wherein said heterocyclic ring contains at least one carbon atom, and
(f) (C₃-C₈)-cycloalkyl;
R¹⁰ and R¹¹ are independently selected from the group consisting of:
(a) hydrogen,
(b) (C₁-C₅)-alkyl,
(c) (C₆-C₁₀)-aryl optionally substituted with one to three substituents selected from the group consisting of:
(c1) halogen,
(c2) (C₁-C₅)alkyl, and
(c3) (C₁-C₅)-alkoxy, and
(d) (C₃-C₈)-cycloalkyl;
R¹² is (C₁-C₅)alkyl;
R¹³ is selected from the group consisting of hydrogen and (C₁-C₅)-alkyl;
R¹⁴ is selected from the group consisting of
(a) (C₁-C₅)-alkyl optionally substituted with substituents (C₁-C₅)-alkoxy, (C₃-C₈)-spiro-cycloalkyl, or (C₆-C₁₀)-aryl optionally substituted with one to three halogens,
(b) (C₆-C₁₀)-aryl optionally substituted with one to three substituents selected from the group consisting of halogen, (C₁-C₅)-alkoxy, and (C₁-C₅)-alkyl optionally substituted with halogen, and
(c) (C₃-C₈)-cycloalkyl optionally substituted with one to three substituents selected from the group consisting of halogen, (C₁-C₅)-alkoxy, and (C₁-C₅)-alkyl optionally substituted with halogen;
R¹⁵ is selected from the group consisting of
(a) hydrogen, and
(b) (C₁-C₅)-alkyl;
R¹⁶ is selected from the group consisting of
(a) hydrogen,
(b) (C₁-C₅)-alkyl, and
(c) (C₃-C₈)cycloalkyl;
or a purified stereoisomer or stereoisomer mixture of said compound, or salt of said compound, stereoisomer or stereoisomer mixture.

3. A pharmaceutical composition for treating or preventing a disease and/or behavior involving the 5-HT₂C receptor which comprises a therapeutically effective amount of a compound of claim 1 and one or more pharmaceutically acceptable ingredients.

4. The pharmaceutical composition of claim 3 which further comprises an additional pharmaceutical agent other than a compound of claim 1, for the treatment or prevention a disease and/or behavior involving the 5-HT_{2C} receptor.

5. The pharmaceutical composition of claim 4 wherein said additional agent is an appetite suppressant selected from the group consisting of benzphetamine, diethylpropion, mazindol, phendimetrazine and phentermine.

6. The pharmaceutical composition of claim 4 wherein said additional agent is an agent for treating obesity related disorders selected from the group consisting of insulin-dependent diabetes, non-insulin dependent diabetes, abnormal feeding behavior, eating disorders and premenstrual tension.

7. The pharmaceutical composition of claim 4 wherein said agent(s) for treating obesity related disorders are selected from the group consisting of insulin, tolbutamide, chlorpropamide, tolazamide, acetohexamide, glycburide, glipizide, gliclazide, tricyclic monoamine oxidase (MAO) inhibitors and serotonin reuptake inhibitors

8. A method of treating or preventing a disease and/or behavior involving the 5-HT_{2C} receptor which comprises administering a therapeutically effective amount of a compound of claim 1 or the composition of claim 3.

9. The method of claim 8 wherein said disease and/or behavior involving the 5-HT_{2C} receptor is selected from the group consisting of obesity, obesity related disorders, abnormal feeding behavior, eating disorders, and premenstrual tension.

10. The method of claim 9 wherein said disease and/or behavior involving the 5-HT_{2C} receptor is obesity.

11. The method of claim 9 wherein said eating disorders are bulimia or anorexia nervosa.

12. A method of treating or preventing a disease correlated to obesity selected from the group consisting of Type II diabetes (NIDDM), hypertension, hyperlipidemia, myocardial infarction and dermatological disease which comprises administering a therapeutically effective amount of a compound of claim 1 or the composition of claim 3.
